(19) United States
(12) Patent Application Publication
(10) Pub. No.: US 2020/0248267 A1
(43) Pub. Date: Aug. 6, 2020

(54) TUMOR VS. MATCHED NORMAL cfRNA

(71) Applicant: NANTOMICS, LLC, Culver City, CA (US)

(72) Inventors: Kathleen DANENBERG, Culver City, CA (US); Shahrooz RABIZADEH, Culver City, CA (US); Patrick SOON-SHIONG, Culver City, CA (US)

(21) Appl. No.: 16/610,476

(22) PCT Filed: May 1, 2018
(86) PCT No.: PCT/US2018/030472

§ 371 Date: Nov. 1, 2019

Related U.S. Application Data
(60) Provisional application No. 62/500,497, filed on May 3, 2017.

(51) Int. Cl. C12Q 1/6886; C12Q 1/686

(52) U.S. Cl. CPC C12Q 1/6886; G01N 2800/52; C12Q 2600/156; C12Q 1/686

(57) ABSTRACT

Compositions and methods for isolation and use of cfRNA are disclosed. Most preferably the cfRNA includes a patient-and tumor-specific mutation, and/or encodes a gene that is relevant in immune response or immune suppression. The identity and/or quantity of cfRNA can be further used for diagnosis of tumor, monitoring of prognosis of the tumor, monitoring the effectiveness of treatment provided to the patients, evaluating a treatment regime based on a likelihood of success of the treatment regime, and even as discovery tool that allows repeated and non-invasive sampling of a patient.

This application claims priority to our co-pending U.S. provisional application having the Ser. No. 62/500,497, filed Mar. May 3, 2017, which is incorporated in its entirety herein.

### FIELD OF THE INVENTION

The field of the invention is analysis of nucleic acids, and especially the use of cell free RNA (cfRNA) to direct, monitor, and/or modify treatment of a patient diagnosed with a tumor.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Cell-free DNA (cfDNA) has been known and characterized from biological fluids over many years, and cfDNA has been employed in efforts to diagnose cancer and monitor response of a cancer to a treatment. More recently, advances in molecular genetics have not only enabled detection of cfDNA at relatively low levels, but also allowed identification of mutated cfDNA. Due to the convenient manner of obtaining cfDNA, analysis of circulating nucleic acids has become an attractive tool in the diagnosis and treatment of cancer. However, cfDNA analysis is somewhat limited in that information obtained does not provide insight about actual translation (i.e., presence of the corresponding protein) or expression level of a gene.

To circumvent at least some the difficulties associated with cfDNA, compositions and methods for detection and analysis of cell free RNA (cfRNA) have recently been developed, and certain methods are found in WO 2016/077709. While detection of cfRNA is desirable from various perspectives, numerous difficulties nevertheless remain. Among other factors, as cfRNA is relatively rare, cfRNA tests need to have significant sensitivity and specificity with respect to a patient's tumor. Such challenge in the analysis of disease (and especially cancer) is still further compounded by the fact that the fraction of circulating tumor RNA (ctRNA) may represent only a small fraction of total cfRNA in the blood or other biological fluids.

To circumvent such difficulties, selected cfRNA tests have focused on detecting already known markers specific to certain tumors. For example, U.S. Pat. No. 9,469,876 to Kuslich and U.S. Pat. No. 8,597,892 to Shelton discuss detecting circulating microRNA biomarkers associated with circulating vesicles in the blood for diagnosis of a specific type of cancer (e.g., prostate cancer, etc.). In another example, U.S. Pat. No. 8,440,396 to Kopreski discloses detection of circulating mRNA fragment of genes encoding tumor associated antigens that are known as markers of several types of cancers (e.g., melanoma, leukemia, etc.). However, such approach is often limited in determining prognosis of cancer patients as not all tumors have universal markers (e.g., HER2, PSA, etc.) that can be conveniently monitored via cfRNA, and as not all patients having same or similar type of cancer may even express same type of marker genes in a same manner.

Therefore, even though numerous methods of implementing, monitoring, and modifying cancer treatment are known in the art, almost all of them suffer from various disadvantages. Thus, there remains a need for improved systems and methods to enable periodic, non-invasive, and highly specific analysis of cfRNA.

### SUMMARY OF THE INVENTION

The inventive subject matter is directed to various compositions and methods for analysis of cfRNA, especially as it relates to implementation, monitoring, and/or change in treatment of a tumor. Contemplated cfRNAs will preferably include cfRNA with patient- and tumor-specific mutations, but also miRNA and other regulatory RNA molecules, including siRNA, shRNA, and intronic RNA that are preferably specific to a tumor.

In one aspect of the inventive subject matter, the inventors contemplate method of monitoring a cancer in a patient that includes a step of identifying a patient- and tumor-specific mutation in a gene of a tumor of the patient. In another step, a bodily fluid is obtained from the patient, and in a still further step a cfRNA that includes the patient- and tumor-specific mutation is quantified in the bodily fluid of the patient.

Most typically, the patient- and tumor-specific mutation in a gene can be identified by comparing one or more omics data from tumor tissue and normal tissue of the same patient. Preferably, the omics data include at least one of whole genome sequence data, exome sequence data, transcriptome sequence data, and/or proteome sequence data. Preferably, the omics data are compared in an incremental synchronous manner. In some embodiments, such identified the patient- and tumor-specific mutation may be used, together with a pathway model (e.g., PARADIGM), to infer a physiological parameter of the tumor (e.g., sensitivity of the tumor to a drug) as well as providing feedback to the pathway model with empirical data.

With respect to the patient- and tumor-specific mutation, it is contemplated that such mutation may encode a neoepitope, which may be derived from a cancer driver gene. Moreover, the patient- and tumor-specific mutation may also be associated with a clonal population of cancer cells within the tumor to so allow for monitoring distinct subsets of cancer cells in the same patient. Of course, it should also be recognized that one or more steps (e.g., the step of obtaining the bodily fluid and the step of quantifying the cfRNA) of contemplated methods may be repeated, during the treatment or before/after treatment. In such case, the step of identifying the patient- and tumor-specific mutation may thus identify a second patient- and tumor-specific mutation in a second gene, which may then be used to quantify a cfRNA that comprises the second patient- and tumor-specific mutation.

In addition, it is generally preferred that the bodily fluid is serum or plasma. In such embodiment, it is also preferred that the step of quantifying includes a step of removing cells from the bodily fluid under conditions and using RNA stabilization agents that substantially avoid cell lysis. Quantification may then be performed using real time quantitative PCR of a cDNA prepared from the cfRNA. Where desired, at least some of the bodily fluid or cfRNA isolated from the bodily fluid or cDNA prepared from the cfRNA may be archived. For example, cfRNA may be frozen at −80° C., while cDNA may be frozen at −4° C. or refrigerated at +2-8° C. Contemplated methods may also include a step of generating or updating a patient record with an indication of prognosis of a tumor that is associated with a quantity of the cfRNA, and/or a step of associating a treatment option and/or a likelihood of success of the treatment option with an amount of quantified cfRNA.

Therefore, and viewed from a different perspective, the inventors also contemplate a method of monitoring a cancer in a patient that includes a step of obtaining a plurality samples of bodily fluids from the patient at a plurality of respective time points, and a further step of quantifying a first cfRNA in each sample of the bodily fluids of the patient, wherein the first cfRNA comprises a first patient- and tumor-specific mutation in a gene of a tumor of the patient.

In some aspects of the inventive subject matter, the contemplated method may further include a step of identifying a second patient- and tumor-specific mutation in a second gene of the tumor of the patient, and another step of quantifying a second cfRNA comprising the second patient- and tumor-specific mutation in the bodily fluid of the patient. Most typically, at least one of the first and second patient- and tumor-specific mutations are identified by comparing omics data from tumor tissue and normal tissue of the same patient (e.g., whole genome sequence data, exome sequence data, transcriptome sequence data, and/or proteome sequence data). Where desired or practical, the omics data are preferably compared by incremental synchronous alignments. Moreover, a pathway model (e.g., PARADIGM) and the patient- and tumor-specific mutation may be used to infer a physiological parameter of the tumor (e.g., sensitivity of the tumor to a drug).

As noted earlier, at least one of the first and second patient- and tumor-specific mutations may encode a neoepitope, and/or be located in a cancer driver gene, and/or may be associated with a clonal population of cancer cells within the tumor. As also noted earlier, the step of obtaining the bodily fluid and the step of quantifying the first and/or second cfRNA may be repeated, typically during and/or before/after providing a treatment regimen to the patient.

In other aspects of the inventive subject matter, such methods may also include a step of identifying a second gene of the tumor of the patient, and a further step of quantifying a second cfRNA derived from the second gene in the bodily fluid of the patient. Preferably, the second gene may be a cancer driver gene, a cancer associated gene, or a cancer specific gene. Alternatively, the second gene may also be a gene that is determined to be overexpressed or under-expressed in the tumor of the patient relative to a normal tissue of the same patient. In still further contemplated aspects, the second gene may be at least one of a checkpoint inhibition related gene, a cytokine related gene, and a chemokine related gene.

In still further aspects of the inventive subject matter, the inventors also contemplate a method of determining a mutational signature in a patient. The method includes a step of quantifying cfRNAs of first and second genes in a bodily fluid of the patient, wherein at least one of the first and second genes comprises a patient- and tumor-specific mutation. Preferably, at least one of patient- and tumor-specific mutation in the first or second gene may encode a neoepitope.

In some embodiments, the first and second genes may be same type of genes. In other embodiments, the first and second genes may be different types of genes. For example, the first gene is a cancer driver gene, while the second gene may be an immune status related gene (e.g., checkpoint inhibition related gene, a gene encoding a cytokine, or a gene encoding a chemokine). Where desired, the step of quantifying the cfRNA may be performed prior to or during treatment (e.g., using a checkpoint inhibitor, an immune therapeutic drug, a chemotherapeutic drug, and/or radiation treatment).

In still another aspect of the inventive subject matter, the inventors contemplate a cfRNA collection kit. The kit comprises a first container (preferably for collection of blood) that includes an RNase inhibitor, a preservative agent, a metabolic inhibitor, and a chelator, wherein the first container is suitable for centrifugation at a relative centrifugal force of 16,000; and a second container (preferably for isolation/purification of cfRNA) that comprises a material that selectively binds or degrades cfDNA.

In a preferred embodiment, the RNase inhibitor may comprise aurintricarboxylic acid, the preservative agent may comprise diazolidinyl urea, the metabolic inhibitor may comprise at least one of glyceraldehyde and sodium fluoride, and/or the chelator may comprise EDTA. Moreover, it is generally preferred that the first container further comprises a serum separator gel, while the second container comprises an RNase-free DNase. In further particularly preferred aspects, first and the second containers are configured to allow robotic processing.

Furthermore, the inventors also contemplate a method of isolating cfRNA. This method includes a step of centrifuging whole blood at a first relative centrifugal force (RCF) to obtain a plasma fraction, a step of centrifuging the plasma fraction at a second RCF to obtain a clarified plasma fraction, and yet another step of subjecting at least a portion of the clarified plasma fraction to a DNA degradation step to degrade ctDNA and genomic DNA (gDNA).

Most typically, the step of centrifuging whole blood is performed in the presence of an RNase inhibitor, a preservative agent, a metabolic inhibitor, and a chelator as noted above. Moreover, it is generally preferred that the step of centrifuging whole blood is performed under conditions that preserve the integrity of cellular components. For example, the first RCF may be between 700 and 2,500 (e.g., 1,600), and/or the second RCF may be between 7,000 and 25,000 (e.g., 16,000). It is contemplated that centrifugation at the first RCF is performed between 15-25 minutes (e.g., 20 minutes) and the centrifugation at the second RCF is performed between 5-15 minutes (e.g., 10 minutes). Where desired or required, cfRNA may be stored at −80° C. and/or cDNA prepared from the cfRNA may be stored at −4° C. or refrigerated at +2-8° C.

Various objects, features, aspects and advantages of the inventive subject matter will become more apparent from the following detailed description of preferred embodiments.

### DETAILED DESCRIPTION

The inventors contemplate that tumor cells and/or some immune cells interacting or surrounding the tumor cells release cell free DNA and/or RNA, and more specifically cell free tumor DNA (ctDNA) and/or RNA (ctRNA), to the patient's bodily fluid, and thus may increase the quantity of the specific ctRNA in the patient's bodily fluid as compared to a healthy individual. Given that, the inventors have now discovered that ctDNA and/or ctRNA, and particularly ctRNA with patient- and tumor-specific mutations, can be employed as a sensitive, selective, and quantitative marker for diagnosis of tumor, monitoring of prognosis of the tumor, monitoring the effectiveness of treatment provided to the patients, evaluating a treatment regime based on a likelihood of success of the treatment regime, and even as discovery tool that allows repeated and non-invasive sampling of a patient. In this context, it should be noted that the total cfRNA will include ctRNA, wherein the ctRNA may have a patient and tumor specific mutation and as such be distinguishable from the corresponding cfRNA of healthy cells, or wherein the ctRNA may be selectively expressed in tumor cells and not be expressed in corresponding healthy cells.

Viewed from a different perspective, the inventors therefore discovered that various nucleic acids, more specifically cfDNAs and/or cfRNAs, may be selected for detection and/or monitoring a particular disease (e.g., tumors, cancer, etc.), disease stage, progress of the disease, treatment response/effectiveness of a treatment regimen in a particular patient, and even anticipating treatment response/effectiveness of a treatment regimen in a particular patient before treatment has started.

Consequently, in one especially preferred aspect of the inventive subject matter, the inventors contemplate a method of monitoring a cancer in a patient using cfDNAs and/or cfRNAs, and especially ctDNAs and/or ctRNAs. In this method, a patient- and tumor-specific mutation in a gene is identified from a tumor of the patient. Then, ctDNA/RNA obtained from bodily fluid of the patient can be analyzed and/or quantified to determine the prognosis of the cancer. Most preferably, the ctDNA/ctRNA includes the patient- and tumor-specific mutation, and/or the ctRNA is exclusively expressed in a tumor cell.

As used herein, the term “tumor” refers to, and is interchangeably used with one or more cancer cells, cancer tissues, malignant tumor cells, or malignant tumor tissue, that can be placed or found in one or more anatomical locations in a human body. It should be noted that the term “patient” as used herein includes both individuals that are diagnosed with a condition (e.g., cancer) as well as individuals undergoing examination and/or testing for the purpose of detecting or identifying a condition. Thus, a patient having a tumor refers to both individuals that are diagnosed with a cancer as well as individuals that are suspected to have a cancer. As used herein, the term “provide” or “providing” refers to and includes any acts of manufacturing, generating, placing, enabling to use, transferring, or making ready to use.

Most typically, the patient- and tumor-specific mutation in the tumor can be identified by high-throughput genome sequencing of a whole genome or a whole exome that allows rapid and specific identification of patient- and tumor- specific mutation in a gene. Preferably, such high-throughput genome sequencing is performed to compare tumor and matched normal (i.e., non-diseased tissue from the same patient) of the whole genome or exome to determine a tumor-specific mutation in a gene, preferably using incremental synchronous alignment as described in U.S. Pat. No. 9,721,062, and/or using RNAseq. In addition, proteomics analysis can be performed, most preferably using quantitative mass spectroscopic methods. In some embodiments, high-throughput genome sequencing is further performed to compare the tumor and the matched healthy individual tissue (e.g., squamous cell of the lung cancer patient and squamous cell of a healthy individual, etc.) to determine a patient-specific mutation. While not limiting to the inventive subject matter, the data format containing sequence information of the tumor and matched normal tissue is in SAM, BAM, GAR, or where differences only are listed, in VCF format.

The inventors contemplate that the patient- and tumor-specific mutations can be present in any genes that may directly or indirectly relate to the function of a tumor cell. Thus, the patient- and tumor-specific mutation may be a known mutation that is known to be commonly associated with development and/or prognosis of a known cancer. However, it is also contemplated that the patient- and tumor-specific mutation may not be a common or known mutation among the patients having the same types of tumor. Thus, the patient- and tumor-specific mutation may be present in a known tumor-associated gene, especially in a cancer-driver gene, or may be present in a gene that is not commonly known to be associated with the specific type of tumor or any types of tumors. Of course, it should be appreciated that the mutations may include one or more of missense or nonsense mutations, insertions, deletions, fusions, and/or translocations, all of which may or may not cause formation of full-length mRNA when transcribed. As used here, a cancer-driver gene refers a gene whose mutation can trigger, cause, or facilitate the transformation of a cell to a tumor cell, or trigger, cause, or facilitate the net cell growth under a specific microenvironmental condition.

For example, the patient- and tumor-specific mutation may be present in tumor-associated genes, especially cancer driver gene, including, but not limited to ABL1, ABL2, ACTB, ACVR1B, AKT1, AKT2, AKT3, ALK, AMER11, APC, AR, ARAF, ARFRP1, ARID1A, ARID1B, ASXL1, ATF1, ATM, ATR, ATRX, AURKA, AURKB, AXIN1, AXL, BAP1, BARD1, BCL2, BCL2L1, BCL2L2, BCL6, BCOR, BCORL1, BLM, BMPR1A, BRAF, BRCA1, BRCA2, BRD4, BRIP1, BTG1, BTK, EMSY, CARD11, CBFB, CBL, CCND1, CCND2, CCND3, CCNE1, CD274, CD79A, CD79B, CDC73, CDH1, CDK12, CDK4, CDK6, CDK8, CDKN1A, CDKN1B, CDKN2A, CDKN2B, CDKN2C, CEA, CEBPA, CHD2, CHD4, CHEK1, CHEK2, CIC, CREBBP, CRKL, CRLF2, CSF1R, CTCF, CTLA4, CTNNA1, CTNNB1, CUL3, CYLD, DAXX, DDR2, DEPTOR, DICER1, DNMT3A, DOT1L, EGFR, EP300, EPCAM, EPHA3, EPHA5, EPHA7, EPHB1, ERBB2, ERBB3, ERBB4, EREG, ERG, ERRFI1, ESR1, EWSR1, EZH2, FAM46C, FANCA, FANCC, FANCD2, FANCE, FANCF, FANCG, FANCL, FAS, FAT1, FBXW7, FGF10, FGF14, FGF19, FGF23, FGF3, FGF4, FGF6, FGFR1, FGFR2, FGFR3, FGFR4, FH, FLCN, FLI1, FLT1, FLT3, FLT4, FOLH1, FOXL2, FOXP1, FRS2, FUBP1, GABRA6, GATA1, GATA2, GATA3, GATA4, GATA6, GID4, GLI1, GNA11, GNA13, GNAQ, GNAS, GPR124, GRIN2A, GRM3, GSK3B, H3F3A, HAVCR2, HGF, HMGB1, HMGB2, HMGB3, HNF1A, HRAS, HSD3B1, HSP90AA1, IDH1, IDH2, IDO, IGF1R, IGF2, IKBKE, IKZF1, IL7R, INHBA, INPP4B, IRF2, IRF4, IRS2, JAK1, JAK2, JAK3, JUN, MYST3, KDM5A, KDM5C, KDM6A, KDR, KEAP, KEL, KIT, KLHL6, KLK3, MLL, MLL2, MLL3, KRAS, LAG3, LMO1, LRP1B, LYN, LZTR1, MAGI2, MAP2K1, MAP2K2, MAP2K4, MAP3K1, MCL1, MDM2, MDM4, MED12, MEF2B, MEN1, MET, MITF, MLH1, MPL, MRE11A, MSH2, MSH6, MTOR, MUC1, MUTYH, MYC, MYCL, MYCN, MYD88, MYH, NF1, NF2, NFE2L2, NFKB1A, NKX2-1, NOTCH1, NOTCH2, NOTCH3, NPM1, NRAS, NSD1, NTRK1, NTRK2, NTRK3, NUP93, PAK3, PALB2, PARK2, PAX3, PAX, PBRM1, PDGFRA, PDCD1, PDCD1LG2, PDGFRB, PDK1, PGR, PIK3C2B, PIK3CA, PIK3CB, PIK3CG, PIK3R1, PIK3R2, PLCG2, PMS2, POLD1, POLE, PPP2R1A, PREX2, PRKAR1A, PRKC1, PRKDC, PRSS8, PTCH1, PTEN, PTPN11, QK1, RAC1, RAD50, RAD51, RAF1, RANBP1, RARA, RB1, RBM10, RET, RICTOR, RIT1, RNF43, ROS1, RPTOR, RUNX1, RUNX1T1, SDHA, SDHB, SDHC, SDHD, SETD2, SF3B1, SLIT2, SMAD2, SMAD3, SMAD4, SMARCA4, SMARCB1, SMO, SNCAIP, SOCS1, SOX10, SOX2, SOX9, SPEN, SPOP, SPTA1, SRC, STAG2, STAT3, STAT4, STK11, SUFU, SYK, T (BRACHYURY), TAF1, TBX3, TERC, TERT, TET2, TGFRB2, TNFAIP3, TNFRSF14, TOP1, TOP2A, TP53, TSC1, TSC2, TSHR, U2AF1, VEGFA, VHL, WISP3, WT1, XPO1, ZBTB2, ZNF217, ZNF703, CD26, CD49F, CD44, CD49F, CD13, CD15, CD29, CD151, CD138, CD166, CD133, CD45, CD90, CD24, CD44, CD38, CD47, CD96, CD 45, CD90, ABCB5, ABCG2, ALCAM, ALPHA-FETOPROTEIN, DLL1, DLL3, DLL4, ENDOGLIN, GJA1, OVASTACIN, AMACR, NESTIN, STRO-1, MICL, ALDH, BMI-1, GLI-2, CXCR1, CXCR2, CX3CR1, CX3CL1, CXCR4, PON1, TROP1, LGR5, MSI-1, C-MAF, TNFRSF7, TNFRSF16, SOX2, PODOPLANIN, L1CAM, HIF-2 ALPHA, TFRC, ERCC1, TUBB3, TOP1, TOP2A, TOP2B, ENOX2, TYMP, TYMS, FOLR1, GPNMB, PAPPA, GART, EBNA1, EBNA2, LMP1, BAGE, BAGE2, BCMA, C10ORF54, CD4, CD8, CD19, CD20, CD25, CD30, CD33, CD80, CD86, CD123, CD276, CCL1, CCL2, CCL3, CCL4, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, CCL28, CCR1, CCR2, CCR3, CCR4, CCR5, CCR6, CCR7, CCR8, CCR9, CCR10, CXCL1, CXCL2, CXCL3, CXCL5, CXCL6, CXCL9, CXCL10, CXCL11, CXCL12, CXCL13, CXCL14, CXCL16, CXCL17, CXCR3, CXCR5, CXCR6, CTAG1B, CTAG2, CTAG1, CTAG4, CTAG5, CTAG6, CTAG9, CAGE1, GAGE1, GAGE2A, GAGE2B, GAGE2C, GAGE2D, GAGE2E, GAGE4, GAGE10, GAGE12D, GAGE12F, GAGE12J, GAGE13, HHLA2, ICOSLG, LAG1, MAGEA10, MAGEA12, MAGEA1, MAGEA2, MAGEA3, MAGEA4, MAGEA4, MAGEA5, MAGEA6, MAGEA7, MAGEA8, MAGEA9, MAGEB1, MAGEB2, MAGEB3, MAGEB4, MAGEB6, MAGEB10, MAGEB16, MAGEB18, MAGEC1, MAGEC2, MAGEC3, MAGED1, MAGED2, MAGED4, MAGED4B, MAGEE1, MAGEE2, MAGEF1, MAGEH1, MAGEL2, NCR3LG1, SLAMF7, SPAG1, SPAG4, SPAG5, SPAG6, SPAG7, SPAG8, SPAG9, SPAG11A, SPAG11B, SPAG16, SPAG17, VTCN1, XAGE1D, XAGE2, XAGE3, XAGE5, XCL1, XCL2, and XCR1.

For another example, some patient- and tumor-specific mutations may be present in genes encoding one or more inflammation-related proteins, including, but not limited to, HMGB1, HMGB2, HMGB3, MUC1, VWF, MMP, CRP, PBEF1, TNF-α, TGF-β, PDGFA, IL-1, IL-2, IL-3, IL-4, IL-5, IL-6, IL-7, IL-8, IL-9, IL-10, IL-12, IL-13, IL-15, IL-17, Eotaxin, FGF, G-CSF, GM-CSF, IFN-γ, IP-10, MCP-1, PDGF, and hTERT, and in yet another example, the ctRNA encoded a full length or a fragment of HMGB1.

For still another example, some patient- and tumor-specific mutations may be present in gene DNA repair-related proteins or RNA repair-related proteins. Table 1 provides an exemplary collection of predominant RNA repair genes and their associated repair pathways contemplated herein, but it should be recognized that numerous other genes associated with DNA repair and repair pathways are also expressly contemplated herein, and Tables 2 and 3 illustrate further exemplary genes for analysis and their associated function in DNA repair.

TABLE 1

| Repair mechanism | Predominant DNA Repair genes |
|---|---|
| Base excision repair | DNA glycosylase, APE1, XRCC1, PNKP, |
| (BER) | Tdp1, APTX, DNA polymerase β, FEN1, |
| | DNA polymerase δ or ε, PCNA-RFC, PARP |
| Mismatch repair | MutSα (MSH2-MSH6), MutSβ (MSH2-MSH3), |
| (MMR) | MutLα (MLH1-PMS2), MutLβ (MLH1-PMS2), |
| | MutLγ (MLH1-MLH3), Exo1, PCNA-RFC |
| Nucleotide excision | XPC-Rad23B-CEN2, UV-DDB (DDB1-XPE), |
| repair (NER) | CSA, CSB, TFIIH, XPB, XPD, XPA, RPA, |
| | XPG, ERCC1-XPF, DNA polymerase δ or ε |
| Homologous | Mre11-Rad50-Nbs1, CtIP, RPA, Rad51, Rad52, |
| recombination (HR) | BRCA1, BRCA2, Exo1, BLM-TopIIIα, |
| | GEN1-Yen1, Slx1-Slx4, Mus81/Eme1 |
| Non-homologous | Ku70-Ku80, DNA-PKc, XRCC4-DNA |
| end-joining (NHEJ) | ligase IV, XLF |

TABLE 2

| Gene name | | Accession |
|---|---|---|
| (synonyms) | Activity | number |
| Base excision | | |
| repair (BER) | | |
| | DNA glycosylases: major altered | |
| | base released | |
| UNG | U excision | NM_003362 |
| SMUG1 | U excision | NM_014311 |
| MBD4 | U or T opposite G at CpG | NM_003925 |
| | sequences | |
| TDG | U, T or ethenoC opposite G | NM_003211 |
| OGG1 | 8-oxoG opposite C | NM_002542 |
| MYH | A opposite 8-oxoG | NM_012222 |
| NTH1 | Ring-saturated or fragmented | NM_002528 |
| | pyrimidines | |
| MPG | 3-meA, ethenoA, hypoxanthine | NM_002434 |
| | Other BER factors | |
| APE1 (HAP1, | AP endonuclease | NM_001641 |
| APEX, REF1) | | |
| APE2 (APEXL2) | AP endonuclease | NM_014481 |
| LIG3 | Main ligation function | NM_013975 |
| XRCC1 | Main ligation function | NM_006297 |
| | Poly(ADP-ribose) polymerase | |
| | (PARP) enzymes | |
| ADPRT | Protects strand interruptions | NM_001618 |
| ADPRTL2 | PARP-like enzyme | NM_005485 |
| ADPRTL3 | PARP-like enzyme | AF085734 |
| Direct reversal of | | |
| damage | | |
| MGMT | O6-meG alkyltransferase | NM_002412 |
| Mismatch excision | | |
| repair (MMR) | | |
| MSH2 | Mismatch and loop recognition | NM_000251 |
| MSH3 | Mismatch and loop recognition | NM_002439 |
| MSH6 | Mismatch recognition | NM_000179 |
| MSH4 | MutS homolog specialized for | NM_002440 |
| | meiosis | |
| MSH5 | MutS homolog specialized for | NM_002441 |
| | meiosis | |
| PMS1 | Mitochondrial MutL homolog | NM_000534 |
| MLH1 | MutL homolog | NM_000249 |
| PMS2 | MutL homolog | NM_000535 |
| MLH3 | MutL homolog of unknown | NM_014381 |
| | function | |
| PMS2L3 | MutL homolog of unknown | D38437 |
| | function | |
| PMS2L4 | MutL homolog of unknown | D38438 |
| | function | |
| Nucleotide excision | | |
| repair (NER) | | |
| XPC | Binds damaged DNA as complex | NM_004628 |
| RAD23B (HR23B) | Binds damaged DNA as complex | NM_002874 |
| CETN2 | Binds damaged DNA as complex | NM_004344 |
| RAD23A (HR23A) | Substitutes for HR23B | NM_005053 |
| XPA | Binds damaged DNA in preincision | NM_000380 |
| | complex | |
| RPA1 | Binds DNA in preincision complex | NM_002945 |
| RPA2 | Binds DNA in preincision complex | NM_002946 |
| RPA3 | Binds DNA in preincision complex | NM_002947 |
| TFIIH | Catalyzes unwinding in preincision | |
| | complex | |
| XPB (ERCC3) | 3′ to 5′ DNA helicase | NM_000122 |
| XPD (ERCC2) | 5′ to 3′ DNA helicase | X52221 |
| GTF2H1 | Core TFIIH subunit p62 | NM_005316 |
| GTF2H2 | Core TFIIH subunit p44 | NM_001515 |
| GTF2H3 | Core TFIIH subunit p34 | NM_001516 |
| GTF2H4 | Core TFIIH subunit p52 | NM_001517 |
| CDK7 | Kinase subunit of TFIIH | NM_001799 |
| CCNH | Kinase subunit of TFIIH | NM_001239 |
| MNAT1 | Kinase subunit of TFIIH | NM_002431 |
| XPG (ERCC5) | 3′ incision | NM_000123 |
| ERCC1 | 5′ incision subunit | NM_001983 |
| XPF (ERCC4) | 5′ incision subunit | NM_005236 |
| LIG1 | DNA joining | NM_000234 |
| NER-related | | |
| CSA (CKN1) | Cockayne syndrome; needed for | NM_000082 |
| | transcription-coupled NER | |
| CSB (ERCC6) | Cockayne syndrome; needed for | NM_000124 |
| | transcription-coupled NER | |
| XAB2 (HCNP) | Cockayne syndrome; needed for | NM_020196 |
| | transcription-coupled NER | |
| DDB1 | Complex defective in XP group E | NM_001923 |
| DDB2 | Mutated in XP group E | NM_000107 |
| MMS19 | Transcription and NER | AW852889 |
| Homologous | | |
| recombination | | |
| RAD51 | Homologous pairing | NM_002875 |
| RAD51L1 | Rad51 homolog | U84138 |
| (RAD51B) | | |
| RAD51C | Rad51 homolog | NM_002876 |
| RAD51L3 | Rad51 homolog | NM_002878 |
| (RAD51D) | | |
| DMC1 | Rad51 homolog, meiosis | NM_007068 |
| XRCC2 | DNA break and cross-link repair | NM_005431 |
| XRCC3 | DNA break and cross-link repair | NM_005432 |
| RAD52 | Accessory factor for recombination | NM_002879 |
| RAD54L | Accessory factor for recombination | NM_003579 |
| RAD54B | Accessory factor for recombination | NM_012415 |
| BRCA1 | Accessory factor for transcription | NM_007295 |
| | and recombination | |
| BRCA2 | Cooperation with RAD51, essential | NM_000059 |
| | function | |
| RAD50 | ATPase in complex with MRE11A, | NM_005732 |
| | NBS1 | |
| MRE11A | 3′ exonuclease | NM_005590 |
| NBS1 | Mutated in Nijmegen breakage | NM_002485 |
| | syndrome | |
| Nonhomologous | | |
| end-joining | | |
| Ku70 (G22P1) | DNA end binding | NM_001469 |
| Ku80 (XRCC5) | DNA end binding | M30938 |
| PRKDC | DNA-dependent protein kinase | NM_006904 |
| | catalytic subunit | |
| LIG4 | Nonhomologous end-joining | NM_002312 |
| XRCC4 | Nonhomologous end-joining | NM_003401 |
| Sanitization of | | |
| nucleotide pools | | |
| MTH1 (NUDT1) | 8-oxoGTPase | NM_002452 |
| DUT | dUTPase | NM_001948 |
| DNA polymerases | | |
| (catalytic subunits) | | |
| POLB | BER in nuclear DNA | NM_002690 |
| POLG | BER in mitochondrial DNA | NM_002693 |
| POLD1 | NER and MMR | NM_002691 |
| POLE1 | NER and MMR | NM_006231 |
| PCNA | Sliding clamp for pol delta | NM_002592 |
| | and pol epsilon | |
| REV3L (POLZ) | DNA pol zeta catalytic subunit, | NM_002912 |
| | essential function | |
| REV7 (MAD2L2) | DNA pol zeta subunit | NM_006341 |
| REV1 | dCMP transferase | NM_016316 |
| POLH | XP variant | NM_006502 |
| POLI (RAD30B) | Lesion bypass | NM_007195 |
| POLQ | DNA cross-link repair | NM_006596 |
| DINB1 (POLK) | Lesion bypass | NM_016218 |
| POLL | Meiotic function | NM_013274 |
| POLM | Presumed specialized lymphoid | NM_013284 |
| | function | |
| TRF4-1 | Sister-chromatid cohesion | AF089896 |
| TRF4-2 | Sister-chromatid cohesion | AF089897 |
| Editing and | | |
| processing | | |
| nucleases | | |
| FEN1 (DNase IV) | 5′ nuclease | NM_004111 |
| TREX1 (DNase III) | 3′ exonuclease | NM_007248 |
| TREX2 | 3′ exonuclease | NM_007205 |
| EX01 (HEX1) | 5′ exonuclease | NM_003686 |
| SPO11 | endonuclease | NM_012444 |
| Rad6 pathway | | |
| UBE2A (RAD6A) | Ubiquitin-conjugating enzyme | NM_003336 |
| UBE2B (RAD6B) | Ubiquitin-conjugating enzyme | NM_003337 |
| RAD18 | Assists repair or replication of | AB035274 |
| | damaged DNA | |
| UBE2VE (MMS2) | Ubiquitin-conjugating complex | AF049140 |
| UBE2N (UBC13, | Ubiquitin-conjugating complex | NM_003348 |
| BTG1) | | |
| Genes defective in | | |
| diseases associated | | |
| with sensitivity | | |
| to DNA | | |
| damaging agents | | |
| BLM | Bloom syndrome helicase | NM_000057 |
| WRN | Werner syndrome helicase/3′- | NM_000553 |
| | exonuclease | |
| RECQL4 | Rothmund-Thompson syndrome | NM_004260 |
| ATM | Ataxia telangiectasia | NM_000051 |
| Fanconi anemia | | |
| FANCA | Involved in tolerance or repair of | NM_000135 |
| | DNA cross-links | |
| FANCB | Involved in tolerance or repair of | N/A |
| | DNA cross-links | |
| FANCC | Involved in tolerance or repair of | NM_000136 |
| | DNA cross-links | |
| FANCD | Involved in tolerance or repair of | N/A |
| | DNA cross-links | |
| FANCE | Involved in tolerance or repair of | NM_021922 |
| | DNA cross-links | |
| FANCF | Involved in tolerance or repair of | AF181994 |
| | DNA cross-links | |
| FANCG (XRCC9) | Involved in tolerance or repair of | NM_004629 |
| | DNA cross-links | |
| Other identified | | |
| genes with a | | |
| suspected DNA | | |
| repair function | | |
| SNM1 (PS02) | DNA cross-link repair | D42045 |
| SNM1B | Related to SNM1 | AL137856 |
| SNM1C | Related to SNM1 | AA315885 |
| RPA4 | Similar to RPA2 | NM_013347 |
| ABH (ALKB) | Resistance to alkylation damage | X91992 |
| PNKP | Converts some DNA breaks to | NM_007254 |
| | ligatable ends | |
| Other conserved | | |
| DNA damage | | |
| response genes | | |
| ATR | ATM- and PI-3K-like essential | NM_001184 |
| | kinase | |
| RAD1 (S. pombe) | PCNA-like DNA damage sensor | NM_002853 |
| homolog | | |
| RAD9 (S. pombe) | PCNA-like DNA damage sensor | NM_004584 |
| homolog | | |
| HUS1 (S. pombe) | PCNA-like DNA damage sensor | NM_004507 |
| homolog | | |
| RAD17 (RAD24) | RFC-like DNA damage sensor | NM_002873 |
| TP53BP1 | BRCT protein | NM_005657 |
| CHEK1 | Effector kinase | NM_001274 |
| CHK2 (Rad53) | Effector kinase | NM_007194 |

TABLE 3

| Gene Name | Gene Title | Biological Activity |
|---|---|---|
| RFC2 | replication factor C (activator 1) | DNA replication |
| | 2, 40 kDa | |
| XRCC6 | X-ray repair complementing | DNA ligation /// DNA repair /// double-strand |
| | defective repair in Chinese | break repair via nonhomologous end-joining |
| | hamster cells 6 (Ku autoantigen, | /// DNA recombination /// positive regulation |
| | 70 kDa) | of transcription, DNA-dependent /// double- |
| | | strand break repair via nonhomologous end- |
| | | joining /// response to DNA damage stimulus |
| | | /// DNA recombination |
| APOBEC | apolipoprotein B mRNA editing | For all of APOBEC1, APOBEC2, |
| | enzyme, catalytic polypeptide- | APOBEC3A-H, and APOBEC4, cytidine |
| | like | deaminases. |
| POLD2 | polymerase (DNA directed), delta | DNA replication /// DNA replication |
| | 2, regulatory subunit 50 kDa | |
| PCNA | proliferating cell nuclear antigen | regulation of progression through cell cycle /// |
| | | DNA replication /// regulation of DNA |
| | | replication /// DNA repair /// cell proliferation |
| | | /// phosphoinositide-mediated signaling /// |
| | | DNA replication |
| RPA1 | replication protein A1, 70 kDa | DNA-dependent DNA replication /// DNA |
| | | repair /// DNA recombination /// DNA |
| | | replication |
| RPA1 | replication protein A1, 70 kDa | DNA-dependent DNA replication /// DNA |
| | | repair /// DNA recombination /// DNA |
| | | replication |
| RPA2 | replication protein A2, 32 kDa | DNA replication /// DNA-dependent DNA |
| | | replication |
| ERCC3 | excision repair cross- | DNA topological change /// transcription- |
| | complementing rodent repair | coupled nucleotide-excision repair /// |
| | deficiency, complementation | transcription /// regulation of transcription, |
| | group 3 (xeroderma | DNA-dependent /// transcription from RNA |
| | pigmentosum group B | polymerase II promoter /// induction of |
| | complementing) | apoptosis /// sensory perception of sound /// |
| | | DNA repair /// nucleotide-excision repair /// |
| | | response to DNA damage stimulus /// DNA |
| | | repair |
| UNG | uracil-DNA glycosylase | carbohydrate metabolism /// DNA repair /// |
| | | base-excision repair /// response to DNA |
| | | damage stimulus /// DNA repair /// DNA |
| | | repair |
| ERCC5 | excision repair cross- | transcription-coupled nucleotide-excision |
| | complementing rodent repair | repair /// nucleotide-excision repair /// sensory |
| | deficiency, complementation | perception of sound /// DNA repair /// |
| | group 5 (xeroderma | response to DNA damage stimulus /// |
| | pigmentosum, complementation | nucleotide-excision repair |
| | group G (Cockayne syndrome)) | |
| MLH1 | mutL homolog 1, colon cancer, | mismatch repair /// cell cycle /// negative |
| | nonpolyposis type 2 (E. coli) | regulation of progression through cell cycle /// |
| | | DNA repair /// mismatch repair /// response to |
| | | DNA damage stimulus |
| LIG1 | ligase I, DNA, ATP-dependent | DNA replication /// DNA repair /// DNA |
| | | recombination /// cell cycle /// morphogenesis |
| | | /// cell division /// DNA repair /// response to |
| | | DNA damage stimulus /// DNA metabolism |
| NBN | nibrin | DNA damage checkpoint /// cell cycle |
| | | checkpoint /// double-strand break repair |
| NBN | nibrin | DNA damage checkpoint /// cell cycle |
| | | checkpoint /// double-strand break repair |
| NBN | nibrin | DNA damage checkpoint /// cell cycle |
| | | checkpoint /// double-strand break repair |
| MSH6 | mutS homolog 6 (E. coli) | mismatch repair /// DNA metabolism /// DNA |
| | | repair /// mismatch repair /// response to DNA |
| | | damage stimulus |
| POLD4 | polymerase (DNA-directed), | DNA replication /// DNA replication |
| | delta 4 | |
| RFC5 | replication factor C (activator 1) | DNA replication /// DNA repair /// DNA |
| | 5, 36.5 kDa | replication |
| RFC5 | replication factor C (activator 1) | DNA replication /// DNA repair /// DNA |
| | 5, 36.5 kDa | replication |
| DDB2 /// | damage-specific DNA binding | nucleotide-excision repair /// regulation of |
| LHX3 | protein 2, 48 kDa /// LIM | transcription, DNA-dependent /// organ |
| | homeobox 3 | morphogenesis /// DNA repair /// response to |
| | | DNA damage stimulus /// DNA repair /// |
| | | transcription /// regulation of transcription |
| POLD1 | polymerase (DNA directed), delta | DNA replication /// DNA repair /// response to |
| | 1, catalytic subunit 125 kDa | UV /// DNA replication |
| FANCG | Fanconi anemia, | cell cycle checkpoint /// DNA repair /// DNA |
| | complementation group G | repair /// response to DNA damage stimulus /// |
| | | regulation of progression through cell cycle |
| POLB | polymerase (DNA directed), beta | DNA-dependent DNA replication /// DNA |
| | | repair /// DNA replication /// DNA repair /// |
| | | response to DNA damage stimulus |
| XRCC1 | X-ray repair complementing | single strand break repair |
| | defective repair in Chinese | |
| | hamster cells 1 | |
| MPG | N-methylpurine-DNA | base-excision repair /// DNA dealkylation /// |
| | glycosylase | DNA repair /// base-excision repair /// |
| | | response to DNA damage stimulus |
| RFC2 | replication factor C (activator 1) | DNA replication |
| | 2, 40 kDa | |
| ERCC1 | excision repair cross- | nucleotide-excision repair /// morphogenesis |
| | complementing rodent repair | /// nucleotide-excision repair /// DNA repair /// |
| | deficiency, complementation | response to DNA damage stimulus |
| | group 1 (includes overlapping | |
| | antisense sequence) | |
| TDG | thymine-DNA glycosylase | carbohydrate metabolism /// base-excision |
| | | repair /// DNA repair /// response to DNA |
| | | damage stimulus |
| TDG | thymine-DNA glycosylase | carbohydrate metabolism /// base-excision |
| | | repair /// DNA repair /// response to DNA |
| | | damage stimulus |
| FANCA | Fanconi anemia, | DNA repair /// protein complex assembly /// |
| | complementation group A /// | DNA repair /// response to DNA damage |
| | Fanconi anemia, | stimulus |
| | complementation group A | |
| RFC4 | replication factor C (activator 1) | DNA replication /// DNA strand elongation /// |
| | 4, 37 kDa | DNA repair /// phosphoinositide-mediated |
| | | signaling /// DNA replication |
| RFC3 | replication factor C (activator 1) | DNA replication /// DNA strand elongation |
| | 3, 38 kDa | |
| RFC3 | replication factor C (activator 1) | DNA replication /// DNA strand elongation |
| | 3, 38 kDa | |
| APEX2 | APEX nuclease | DNA repair /// response to DNA damage |
| | (apurinic/apyrimidinic | stimulus |
| | endonuclease) 2 | |
| RAD1 | RAD1 homolog (S. pombe) | DNA repair /// cell cycle checkpoint /// cell |
| | | cycle checkpoint /// DNA damage checkpoint |
| | | /// DNA repair /// response to DNA damage |
| | | stimulus /// meiotic prophase I |
| RAD1 | RAD1 homolog (S. pombe) | DNA repair /// cell cycle checkpoint /// cell |
| | | cycle checkpoint /// DNA damage checkpoint |
| | | /// DNA repair /// response to DNA damage |
| | | stimulus /// meiotic prophase I |
| BRCA1 | breast cancer 1, early onset | regulation of transcription from RNA |
| | | polymerase II promoter /// regulation of |
| | | transcription from RNA polymerase III |
| | | promoter /// DNA damage response, signal |
| | | transduction by p53 class mediator resulting in |
| | | transcription of p21 class mediator/// cell |
| | | cycle /// protein ubiquitination /// androgen |
| | | receptor signaling pathway /// regulation of |
| | | cell proliferation /// regulation of apoptosis /// |
| | | positive regulation of DNA repair /// negative |
| | | regulation of progression through cell cycle /// |
| | | positive regulation of transcription, DNA- |
| | | dependent /// negative regulation of centriole |
| | | replication /// DNA damage response, signal |
| | | transduction resulting in induction of |
| | | apoptosis /// DNA repair /// response to DNA |
| | | damage stimulus /// protein ubiquitination /// |
| | | DNA repair /// regulation of DNA repair /// |
| | | apoptosis /// response to DNA damage |
| | | stimulus |
| EXO1 | exonuclease 1 | DNA repair /// DNA repair /// mismatch repair |
| | | /// DNA recombination |
| FEN1 | flap structure-specific | DNA replication /// double-strand break repair |
| | endonuclease 1 | /// UV protection /// phosphoinositide- |
| | | mediated signaling /// DNA repair /// DNA |
| | | replication /// DNA repair /// DNA repair |
| FEN1 | flap structure-specific | DNA replication /// double-strand break repair |
| | endonuclease 1 | /// UV protection /// phosphoinositide- |
| | | mediated signaling /// DNA repair /// DNA |
| | | replication /// DNA repair /// DNA repair |
| MLH3 | mutL homolog 3 (E. coli) | mismatch repair /// meiotic recombination /// |
| | | DNA repair /// mismatch repair /// response to |
| | | DNA damage stimulus /// mismatch repair |
| MGMT | O-6-methylguanine-DNA | DNA ligation /// DNA repair /// response to |
| | methyltransferase | DNA damage stimulus |
| RAD51 | RAD51 homolog (RecA | double-strand break repair via homologous |
| | homolog, E. coli) (S. cerevisiae) | recombination /// DNA unwinding during |
| | | replication /// DNA repair /// mitotic |
| | | recombination /// meiosis /// meiotic |
| | | recombination /// positive regulation of DNA |
| | | ligation /// protein homo-oligomerization /// |
| | | response to DNA damage stimulus /// DNA |
| | | metabolism /// DNA repair /// response to |
| | | DNA damage stimulus /// DNA repair /// DNA |
| | | recombination /// meiotic recombination /// |
| | | double-strand break repair via homologous |
| | | recombination /// DNA unwinding during |
| | | replication |
| RAD51 | RAD51 homolog (RecA | double-strand break repair via homologous |
| | homolog, E. coli) (S. cerevisiae) | recombination /// DNA unwinding during |
| | | replication /// DNA repair /// mitotic |
| | | recombination /// meiosis /// meiotic |
| | | recombination /// positive regulation of DNA |
| | | ligation /// protein homo-oligomerization /// |
| | | response to DNA damage stimulus /// DNA |
| | | metabolism /// DNA repair /// response to |
| | | DNA damage stimulus /// DNA repair /// DNA |
| | | recombination /// meiotic recombination /// |
| | | double-strand break repair via homologous |
| | | recombination /// DNA unwinding during |
| | | replication |
| XRCC4 | X-ray repair complementing | DNA repair /// double-strand break repair /// |
| | defective repair in Chinese | DNA recombination /// DNA recombination |
| | hamster cells 4 | /// response to DNA damage stimulus |
| XRCC4 | X-ray repair complementing | DNA repair /// double-strand break repair /// |
| | defective repair in Chinese | DNA recombination /// DNA recombination |
| | hamster cells 4 | /// response to DNA damage stimulus |
| RECQL | RecQ protein-like (DNA helicase | DNA repair /// DNA metabolism |
| | Q1-like) | |
| ERCC8 | excision repair cross- | DNA repair /// transcription /// regulation of |
| | complementing rodent repair | transcription, DNA-dependent /// sensory |
| | deficiency, complementation | perception of sound /// transcription-coupled |
| | group 8 | nucleotide-excision repair |
| FANCC | Fanconi anemia, | DNA repair /// DNA repair /// protein complex |
| | complementation group C | assembly /// response to DNA damage |
| | | stimulus |
| OGG1 | 8-oxoguanine DNA glycosylase | carbohydrate metabolism /// base-excision |
| | | repair /// DNA repair /// base-excision repair |
| | | /// response to DNA damage stimulus /// DNA |
| | | repair |
| MRE11A | MRE11 meiotic recombination | regulation of mitotic recombination /// double- |
| | 11 homolog A (S. cerevisiae) | strand break repair via nonhomologous end- |
| | | joining /// telomerase-dependent telomere |
| | | maintenance /// meiosis /// meiotic |
| | | recombination /// DNA metabolism /// DNA |
| | | repair /// double-strand break repair /// |
| | | response to DNA damage stimulus /// DNA |
| | | repair /// double-strand break repair /// DNA |
| | | recombination |
| RAD52 | RAD52 homolog (S. cerevisiae) | double-strand break repair /// mitotic |
| | | recombination /// meiotic recombination /// |
| | | DNA repair /// DNA recombination /// |
| | | response to DNA damage stimulus |
| WRN | Werner syndrome | DNA metabolism /// aging |
| XPA | xeroderma pigmentosum, | nucleotide-excision repair /// DNA repair /// |
| | complementation group A | response to DNA damage stimulus /// DNA |
| | | repair /// nucleotide-excision repair |
| BLM | Bloom syndrome | DNA replication /// DNA repair /// DNA |
| | | recombination /// antimicrobial humoral |
| | | response (sensu Vertebrata) /// DNA |
| | | metabolism /// DNA replication |
| OGG1 | 8-oxoguanine DNA glycosylase | carbohydrate metabolism /// base-excision |
| | | repair /// DNA repair /// base-excision repair |
| | | /// response to DNA damage stimulus /// DNA |
| | | repair |
| MSH3 | mutS homolog 3 (E. coli) | mismatch repair /// DNA metabolism /// DNA |
| | | repair /// mismatch repair /// response to DNA |
| | | damage stimulus |
| POLE2 | polymerase (DNA directed), | DNA replication /// DNA repair /// DNA |
| | epsilon 2 (p59 subunit) | replication |
| RAD51C | RAD51 homolog C | DNA repair/// DNA recombination /// DNA |
| | (S. cerevisiae) | metabolism /// DNA repair /// DNA |
| | | recombination /// response to DNA damage |
| | | stimulus |
| LIG4 | ligase IV, DNA, ATP-dependent | single strand break repair /// DNA replication |
| | | /// DNA recombination /// cell cycle /// cell |
| | | division /// DNA repair /// response to DNA |
| | | damage stimulus |
| ERCC6 | excision repair cross- | DNA repair /// transcription /// regulation of |
| | complementing rodent repair | transcription, DNA-dependent /// transcription |
| | deficiency, complementation | from RNA polymerase II promoter /// sensory |
| | group 6 | perception of sound |
| LIG3 | ligase III, DNA, ATP-dependent | DNA replication /// DNA repair /// cell cycle |
| | | /// meiotic recombination /// spermatogenesis |
| | | /// cell division /// DNA repair /// DNA |
| | | recombination /// response to DNA damage |
| | | stimulus |
| RAD17 | RAD17 homolog (S. pombe) | DNA replication /// DNA repair /// cell cycle |
| | | /// response to DNA damage stimulus |
| XRCC2 | X-ray repair complementing | DNA repair /// DNA recombination /// meiosis |
| | defective repair in Chinese | /// DNA metabolism /// DNA repair /// |
| | hamster cells 2 | response to DNA damage stimulus |
| MUTYH | mutY homolog (E. coli) | carbohydrate metabolism /// base-excision |
| | | repair /// mismatch repair /// cell cycle /// |
| | | negative regulation of progression through cell |
| | | cycle /// DNA repair /// response to DNA |
| | | damage stimulus /// DNA repair |
| RFC1 | replication factor C (activator 1) | DNA-dependent DNA replication /// |
| | 1, 145 kDa /// replication factor C | transcription /// regulation of transcription, |
| | (activator 1) 1, 145kDa | DNA-dependent /// telomerase-dependent |
| | | telomere maintenance /// DNA replication /// |
| | | DNA repair |
| RFC1 | replication factor C (activator 1) | DNA-dependent DNA replication /// |
| | 1, 145 kDa | transcription /// regulation of transcription, |
| | | DNA-dependent /// telomerase-dependent |
| | | telomere maintenance /// DNA replication /// |
| | | DNA repair |
| BRCA2 | breast cancer 2, early onset | regulation of progression through cell cycle /// |
| | | double-strand break repair via homologous |
| | | recombination /// DNA repair /// establishment |
| | | and/or maintenance of chromatin architecture |
| | | /// chromatin remodeling /// regulation of S |
| | | phase of mitotic cell cycle /// mitotic |
| | | checkpoint /// regulation of transcription /// |
| | | response to DNA damage stimulus |
| RAD50 | RAD50 homolog (S. cerevisiae) | regulation of mitotic recombination /// double- |
| | | strand break repair /// telomerase-dependent |
| | | telomere maintenance /// cell cycle /// meiosis |
| | | /// meiotic recombination /// chromosome |
| | | organization and biogenesis /// telomere |
| | | maintenance /// DNA repair /// response to |
| | | DNA damage stimulus /// DNA repair /// DNA |
| | | recombination |
| DDB1 | damage-specific DNA binding | nucleotide-excision repair /// ubiquitin cycle |
| | protein 1, 127 kDa | /// DNA repair /// response to DNA damage |
| | | stimulus /// DNA repair |
| XRCC5 | X-ray repair complementing | double-strand break repair via nonhomologous |
| | defective repair in Chinese | end-joining /// DNA recombination /// DNA |
| | hamster cells 5 (double-strand- | repair /// DNA recombination /// response to |
| | break rejoining; Ku autoantigen, | DNA damage stimulus /// double-strand break |
| | 80 kDa) | repair |
| XRCC5 | X-ray repair complementing | double-strand break repair via nonhomologous |
| | defective repair in Chinese | end-joining /// DNA recombination /// DNA |
| | hamster cells 5 (double-strand- | repair /// DNA recombination /// response to |
| | break rejoining; Ku autoantigen, | DNA damage stimulus /// double-strand break |
| | 80 kDa) | repair |
| PARP 1 | poly (ADP-ribose) polymerase | DNA repair /// transcription from RNA |
| | family, member 1 | polymerase II promoter /// protein amino acid |
| | | ADP-ribosylation /// DNA metabolism /// |
| | | DNA repair /// protein amino acid ADP- |
| | | ribosylation /// response to DNA damage |
| | | stimulus |
| POLE3 | polymerase (DNA directed), | DNA replication |
| | epsilon 3 (p17 subunit) | |
| RFC1 | replication factor C (activator 1) | DNA-dependent DNA replication /// |
| | 1, 145 kDa | transcription /// regulation of transcription, |
| | | DNA-dependent /// telomerase-dependent |
| | | telomere maintenance /// DNA replication /// |
| | | DNA repair |
| RAD50 | RAD50 homolog (S. cerevisiae) | regulation of mitotic recombination /// double- |
| | | strand break repair /// telomerase-dependent |
| | | telomere maintenance /// cell cycle /// meiosis |
| | | /// meiotic recombination /// chromosome |
| | | organization and biogenesis /// telomere |
| | | maintenance /// DNA repair /// response to |
| | | DNA damage stimulus /// DNA repair /// DNA |
| | | recombination |
| XPC | xeroderma pigmentosum, | nucleotide-excision repair /// DNA repair /// |
| | complementation group C | nucleotide-excision repair /// response to DNA |
| | | damage stimulus /// DNA repair |
| MSH2 | mutS homolog 2, colon cancer, | mismatch repair /// post-replication repair /// |
| | nonpolyposis type 1 (E. coli) | cell cycle /// negative regulation of |
| | | progression through cell cycle /// DNA |
| | | metabolism /// DNA repair /// mismatch repair |
| | | /// response to DNA damage stimulus /// DNA |
| | | repair |
| RPA3 | replication protein A3, 14 kDa | DNA replication /// DNA repair /// DNA |
| | | replication |
| MBD4 | methyl-CpG binding domain | base-excision repair /// DNA repair /// |
| | protein 4 | response to DNA damage stimulus /// DNA |
| | | repair |
| MBD4 | methyl-CpG binding domain | base-excision repair /// DNA repair /// |
| | protein 4 | response to DNA damage stimulus /// DNA |
| | | repair |
| NTHL1 | nth endonuclease III-like 1 | carbohydrate metabolism /// base-excision |
| | (E. coli) | repair /// nucleotide-excision repair, DNA |
| | | incision, 5′-to lesion /// DNA repair /// |
| | | response to DNA damage stimulus |
| PMS2 /// | PMS2 post-meiotic segregation | mismatch repair /// cell cycle /// negative |
| PMS2CL | increased 2 (S. cerevisiae) /// | regulation of progression through cell cycle /// |
| | PMS2-C terminal-like | DNA repair /// mismatch repair /// response to |
| | | DNA damage stimulus /// mismatch repair |
| RAD51C | RAD51 homolog C | DNA repair /// DNA recombination /// DNA |
| | (S. cerevisiae) | metabolism /// DNA repair /// DNA |
| | | recombination /// response to DNA damage |
| | | stimulus |
| UNG2 | uracil-DNA glycosylase 2 | regulation of progression through cell cycle /// |
| | | carbohydrate metabolism /// base-excision |
| | | repair /// DNA repair /// response to DNA |
| | | damage stimulus |
| APEX1 | APEX nuclease (multifunctional | base-excision repair /// transcription from |
| | DNA repair enzyme) 1 | RNA polymerase II promoter /// regulation of |
| | | DNA binding /// DNA repair /// response to |
| | | DNA damage stimulus |
| ERCC4 | excision repair cross- | nucleotide-excision repair /// nucleotide- |
| | complementing rodent repair | excision repair /// DNA metabolism /// DNA |
| | deficiency, complementation | repair /// response to DNA damage stimulus |
| | group 4 | |
| RAD1 | RAD1 homolog (S. pombe) | DNA repair /// cell cycle checkpoint /// cell |
| | | cycle checkpoint /// DNA damage checkpoint |
| | | /// DNA repair /// response to DNA damage |
| | | stimulus /// meiotic prophase I |
| RECQL5 | RecQ protein-like 5 | DNA repair /// DNA metabolism /// DNA |
| | | metabolism |
| MSH5 | mutS homolog 5 (E. coli) | DNA metabolism /// mismatch repair /// |
| | | mismatch repair /// meiosis /// meiotic |
| | | recombination /// meiotic prophase II /// |
| | | meiosis |
| RECQL | RecQ protein-like (DNA helicase | DNA repair /// DNA metabolism |
| | Q1-like) | |
| RAD52 | RAD52 homolog (S. cerevisiae) | double-strand break repair /// mitotic |
| | | recombination /// meiotic recombination /// |
| | | DNA repair /// DNA recombination /// |
| | | response to DNA damage stimulus |
| XRCC4 | X-ray repair complementing | DNA repair /// double-strand break repair /// |
| | defective repair in Chinese | DNA recombination /// DNA recombination |
| | hamster cells 4 | /// response to DNA damage stimulus |
| XRCC4 | X-ray repair complementing | DNA repair /// double-strand break repair /// |
| | defective repair in Chinese | DNA recombination /// DNA recombination |
| | hamster cells 4 | /// response to DNA damage stimulus |
| RAD17 | RAD17 homolog (S. pombe) | DNA replication /// DNA repair /// cell cycle |
| | | /// response to DNA damage stimulus |
| MSH3 | mutS homolog 3 (E. coli) | mismatch repair /// DNA metabolism /// DNA |
| | | repair /// mismatch repair /// response to DNA |
| | | damage stimulus |
| MRE11A | MRE11 meiotic recombination | regulation of mitotic recombination /// double- |
| | 11 homolog A (S. cerevisiae) | strand break repair via nonhomologous end- |
| | | joining /// telomerase-dependent telomere |
| | | maintenance /// meiosis /// meiotic |
| | | recombination /// DNA metabolism /// DNA |
| | | repair /// double-strand break repair /// |
| | | response to DNA damage stimulus /// DNA |
| | | repair /// double-strand break repair /// DNA |
| | | recombination |
| MSH6 | mutS homolog 6 (E. coli) | mismatch repair /// DNA metabolism /// DNA |
| | | repair /// mismatch repair /// response to DNA |
| | | damage stimulus |
| MSH6 | mutS homolog 6 (E. coli) | mismatch repair /// DNA metabolism /// DNA |
| | | repair /// mismatch repair /// response to DNA |
| | | damage stimulus |
| RECQL5 | RecQ protein-like 5 | DNA repair /// DNA metabolism /// DNA |
| | | metabolism |
| BRCA1 | breast cancer 1, early onset | regulation of transcription from RNA |
| | | polymerase II promoter /// regulation of |
| | | transcription from RNA polymerase III |
| | | promoter /// DNA damage response, signal |
| | | transduction by p53 class mediator resulting in |
| | | transcription of p21 class mediator/// cell |
| | | cycle /// protein ubiquitination /// androgen |
| | | receptor signaling pathway /// regulation of |
| | | cell proliferation /// regulation of apoptosis /// |
| | | positive regulation of DNA repair /// negative |
| | | regulation of progression through cell cycle /// |
| | | positive regulation of transcription, DNA- |
| | | dependent /// negative regulation of centriole |
| | | replication /// DNA damage response, signal |
| | | transduction resulting in induction of |
| | | apoptosis /// DNA repair /// response to DNA |
| | | damage stimulus /// protein ubiquitination /// |
| | | DNA repair /// regulation of DNA repair /// |
| | | apoptosis /// response to DNA damage |
| | | stimulus |
| RAD52 | RAD52 homolog (S. cerevisiae) | double-strand break repair /// mitotic |
| | | recombination /// meiotic recombination /// |
| | | DNA repair /// DNA recombination /// |
| | | response to DNA damage stimulus |
| POLD3 | polymerase (DNA-directed), | DNA synthesis during DNA repair /// |
| | delta 3, accessory subunit | mismatch repair /// DNA replication |
| MSH5 | mutS homolog 5 (E. coli) | DNA metabolism /// mismatch repair /// |
| | | mismatch repair /// meiosis /// meiotic |
| | | recombination /// meiotic prophase II /// |
| | | meiosis |
| ERCC2 | excision repair cross- | transcription-coupled nucleotide-excision |
| | complementing rodent repair | repair /// transcription /// regulation of |
| | deficiency, complementation | transcription, DNA-dependent /// transcription |
| | group 2 (xeroderma | from RNA polymerase II promoter /// |
| | pigmentosum D) | induction of apoptosis /// sensory perception |
| | | of sound /// nucleobase, nucleoside, nucleotide |
| | | and nucleic acid metabolism /// nucleotide- |
| | | excision repair |
| RECQL4 | RecQ protein-like 4 | DNA repair /// development /// DNA |
| | | metabolism |
| PMS1 | PMS1 post-meiotic segregation | mismatch repair /// regulation of transcription, |
| | increased 1 (S. cerevisiae) | DNA-dependent /// cell cycle /// negative |
| | | regulation of progression through cell cycle /// |
| | | mismatch repair /// DNA repair /// response to |
| | | DNA damage stimulus |
| ZFP276 | zinc finger protein 276 homolog | transcription /// regulation of transcription, |
| | (mouse) | DNA-dependent |
| MBD4 | methyl-CpG binding domain | base-excision repair /// DNA repair /// |
| | protein 4 | response to DNA damage stimulus /// DNA |
| | | repair |
| MBD4 | methyl-CpG binding domain | base-excision repair /// DNA repair /// |
| | protein 4 | response to DNA damage stimulus /// DNA |
| | | repair |
| MLH3 | mutL homolog 3 (E. coli) | mismatch repair /// meiotic recombination /// |
| | | DNA repair /// mismatch repair /// response to |
| | | DNA damage stimulus /// mismatch repair |
| FANCA | Fanconi anemia, | DNA repair /// protein complex assembly /// |
| | complementation group A | DNA repair /// response to DNA damage |
| | | stimulus |
| POLE | polymerase (DNA directed), | DNA replication /// DNA repair /// DNA |
| | epsilon | replication /// response to DNA damage |
| | | stimulus |
| XRCC3 | X-ray repair complementing | DNA repair /// DNA recombination /// DNA |
| | defective repair in Chinese | metabolism /// DNA repair /// DNA |
| | hamster cells 3 | recombination /// response to DNA damage |
| | | stimulus /// response to DNA damage stimulus |
| MLH3 | mutL homolog 3 (E. coli) | mismatch repair /// meiotic recombination /// |
| | | DNA repair /// mismatch repair /// response to |
| | | DNA damage stimulus /// mismatch repair |
| NBN | nibrin | DNA damage checkpoint /// cell cycle |
| | | checkpoint /// double-strand break repair |
| SMUG1 | single-strand selective | carbohydrate metabolism /// DNA repair /// |
| | monofunctional uracil | DNA response to DNA damage stimulus |
| | glycosylase | |
| FANCF | Fanconi anemia, | DNA repair /// response to DNA damage |
| | complementation group F | stimulus |
| NEIL1 | nei endonuclease VIII-like 1 | carbohydrate metabolism /// DNA repair /// |
| | (E. coli) | response to DNA damage stimulus |
| FANCE | Fanconi anemia, | DNA repair /// response to DNA damage |
| | complementation group E | stimulus |
| MSH5 | mutS homolog 5 (E. coli) | DNA metabolism /// mismatch repair /// |
| | | mismatch repair /// meiosis /// meiotic |
| | | recombination /// meiotic prophase II /// |
| | | meiosis |
| RECQL5 | RecQ protein-like 5 | DNA repair /// DNA metabolism /// DNA |
| | | metabolism |

In yet another example, some patient- and tumor-specific mutations may be present in a gene not associated with a disease (e.g., housekeeping genes), which include those related to transcription factors (e.g., ATF1, ATF2, ATF4, ATF6, ATF7, ATFIP, BTF3, E2F4, ERH, HMGB1, ILF2, IER2, JUND, TCEB2, etc.), repressors (e.g., PUF60), RNA splicing (e.g., BAT1, HNRPD, HNRPK, PABPN1, SRSF3, etc.), translation factors (EIF1, EIF1AD, EIF1B, EIF2A, EIF2AK1, EIF2AK3, EIF2AK4, EIF2B2, EIF2B3, EIF2B4, EIF2S2, EIF3A, etc.), tRNA synthetases (e.g., AARS, CARS, DARS, FARS, GARS, HARS, IARS, KARS, MARS, etc.), RNA binding protein (e.g., ELAVL1, etc.), ribosomal proteins (e.g., RPL5, RPL8, RPL9, RPL10, RPL11, RPL14, RPL25, etc.), mitochondrial ribosomal proteins (e.g., MRPL9, MRPL1, MRPL10, MRPL11, MRPL12, MRPL13, MRPL14, etc.), RNA polymerase (e.g., POLR1C, POLR1D, POLR1E, POLR2A, POLR2B, POLR2C, POLR2D, POLR3C, etc.), protein processing (e.g., PPID, PPI3, PPIF, CANX, CAPN1, NACA, PFDN2, SNX2, SS41, SUMO1, etc.), heat shock proteins (e.g., HSPA4, HSPA5, HSBP1, etc.), histone (e.g., HIST1HSBC, H1FX, etc.), cell cycle (e.g., ARHGAP35, RAB10, RAB11A, CCNY, CCNL, PPP1CA, RAD1, RAD17, etc.), carbohydrate metabolism (e.g., ALDOA, GSK3A, PGK1, PGAM5, etc.), lipid metabolism (e.g., HADHA), citric acid cycle (e.g., SDHA, SDHB, etc.), amino acid metabolism (e.g., COMT, etc.), NADH dehydrogenase (e.g., NDUFA2, etc.), cytochrome c oxidase (e.g., COX5B, COX8, COX11, etc.), ATPase (e.g. ATP2C1, ATP5F1, etc.), lysosome (e.g., CTSD, CSTB, LAMP1, etc.), proteasome (e.g., PSMA1, UBA1, etc.), cytoskeletal proteins (e.g., ANXA6, ARPC2, etc.), and organelle synthesis (e.g., BLOC1S1, AP2A1, etc.).

With respect to the type of mutation, it is generally preferred that the patient- and tumor-specific mutation is present in a coding region of a gene (e.g., exome) such that the mutation may affect the amino acid sequence of a protein encoded by the gene. Thus, in some embodiments, the patient- and tumor-specific mutation may result in the generation of tumor- and patient-specific neoepitopes. Most typically, the patient-specific epitopes are unique to the patient, and may as such generate a unique and patient specific marker of a diseased cell or cell population (e.g., sub-clonal fraction of a tumor). Consequently, it should be especially appreciated that ctRNA carrying such patient and tumor specific mutation may be followed as a proxy marker not only for the presence of a tumor, but also for a cell of a specific tumor sub-clone (e.g., treatment resistant tumor). Moreover, where the mutation encodes a patient and tumor specific neoepitope that is used as a target in immune therapy, such the ctRNA carrying such mutation will be able to serve as a highly specific marker for the treatment efficacy of the immune therapy.

Alternatively, it is also contemplated that the patient- and tumor-specific mutation is present in a noncoding region of a gene (e.g., intron, promoter, etc.) such that the mutation may affect the expression level or transcription pattern (e.g., alternative splicing, etc.) of the gene without affecting the amino acid sequence of a protein encoded by the gene. In some embodiments, the patient- and tumor-specific mutation may be present in a gene generating noncoding RNAs (e.g., microRNA, small interfering RNA, long non-coding RNA (IncRNA)) such that the activity or the function of the noncoding RNA may be affected by the mutation.

The inventors contemplate that the patient- and tumor-specific mutation in a gene of the tumor cell can be detected in one or more ctDNA and/or ctRNA obtained from the patient's bodily fluid. In addition, it is also contemplated that some patient- and tumor-specific mutations may affect the expression level of the gene having the patient- and tumor-specific mutation or the expression level of another gene that is downstream of the signaling cascade or that interacts with the gene having the patient- and tumor-specific mutation. In some embodiments, the gene whose expression level is affected may be located in the same cell (e.g., tumor cell). For example, where the another patient- and tumor-specific mutation is located in gene A in the tumor cell that encodes a protein kinase, the expression level of gene A may be affected to reduce or increase the amount of mRNA transcripts of gene A. In still another example, where the patient- and tumor-specific mutation is located in gene A in the tumor cell that encodes a protein kinase, the expression level of gene B may be affected in the same cell as the gene B expression is dependent on the phosphorylation activity by the protein kinase. For still other example, where the patient- and tumor-specific mutation is located in gene A in the tumor cell that encodes a protein kinase, the expression of gene C may be affected in different type of cell (e.g., NKT cell, etc.) upon interaction with a encoded protein by gene B having the mutation. Thus, the patient- and tumor-specific mutation in a gene of the tumor cell may directly or indirectly affect the quantity of ctRNA of the gene with the mutation, ctRNA of another gene, or other cell free RNA of any other gene(s) derived from a cell other than the tumor cell.

Most typically, suitable tissue sources include whole blood, which is preferably provided as plasma or serum. Thus, in a preferred embodiment, the ctDNA and/or ctRNA is isolated from a whole blood sample that is processed under conditions that preserve cellular integrity and stability of ctRNA. Alternatively, it should be noted that various other bodily fluids are also deemed appropriate so long as ctDNA and/or ctRNA is present in such fluids. Appropriate fluids include saliva, ascites fluid, spinal fluid, urine, or any other types of bodily fluid, which may be fresh, chemically preserved, refrigerated or frozen.

The bodily fluid of the patient can be obtained at any desired time point(s) depending on the purpose of the omics analysis. For example, the bodily fluid of the patient can be obtained before and/or after the patient is confirmed to have a tumor and/or periodically thereafter (e.g., every week, every month, etc.) in order to associate the ctDNA and/or ctRNA data with the prognosis of the cancer. In some embodiments, the bodily fluid of the patient can be obtained from a patient before and after the cancer treatment (e.g., chemotherapy, radiotherapy, drug treatment, cancer immunotherapy, etc.). While it may vary depending on the type of treatments and/or the type of cancer, the bodily fluid of the patient can be obtained at least 24 hours, at least 3 days, at least 7 days after the cancer treatment. For more accurate comparison, the bodily fluid from the patient before the cancer treatment can be obtained less than 1 hour, less than 6 hours before, less than 24 hours before, less than a week before the beginning of the cancer treatment. In addition, a plurality of samples of the bodily fluid of the patient can be obtained during a period before and/or after the cancer treatment (e.g., once a day after 24 hours for 7 days, etc.).

Additionally or alternatively, the bodily fluid of a healthy individual can be obtained to compare the sequence/modification of ctDNA and/or ctRNA sequence, and/or quantity/subtype expression of the ctRNA. As used herein, a healthy individual refers an individual without a tumor. Preferably, the healthy individual can be chosen among group of people shares characteristics with the patient (e.g., age, gender, ethnicity, diet, living environment, family history, etc.).

Any suitable methods for isolating cell free DNA/RNA are contemplated. For example, in one exemplary method of DNA isolation, specimens were accepted as 10 ml of whole blood drawn into a test tube. Cell free DNA can be isolated from other from mono-nucleosomal and di-nucleosomal complexes using magnetic beads that can separate out cell free DNA at a size between 100-300 bps. For another example, in one exemplary method of RNA isolation, specimens were accepted as 10 ml of whole blood drawn into cell-free RNA BCT® tubes or cell-free DNA BCT® tubes containing RNA stabilizers, respectively. Advantageously, cell free RNA is stable in whole blood in the cell-free RNA BCT tubes for seven days while cell free RNA is stable in whole blood in the cell-free DNA BCT Tubes for fourteen days, allowing time for shipping of patient samples from world-wide locations without the degradation of cell free RNA.

It is generally preferred that the cfRNA is isolated using RNA stabilization reagents. While any suitable RNA stabilization agents are contemplated, preferred RNA stabilization reagents include one or more of a nuclease inhibitor, a preservative agent, a metabolic inhibitor, and/or a chelator. For example, contemplated nuclease inhibitors may include RNAase inhibitors such as diethyl pyrocarbonate, ethanol, aurintricarboxylic acid (ATA), formamide, vanadyl-ribonucleoside complexes, macaloid, heparin, bentonite, ammonium sulfate, dithiothreitol (DTT), beta-mercaptoethanol, dithioerythritol, tris(2-carboxyethyl)phosphene hydrochloride, most typically in an amount of between 0.5 to 2.5 wt %. Preservative agents may include diazolidinyl urea (DU), imidazolidinyl urea, dimethoylol-5,5-dimethylhydantoin, dimethylol urea, 2-bromo-2-nitropropane-1,3-diol, oxazolidines, sodium hydroxymethyl glycinate, 5-hydroxymethoxymethyl-1-laza-3,7-dioxabicyclo[3.3.0]octane, 5-hydroxymethyl-1-laza-3,7dioxabicyclo[3.3.0]octane, 5-hydroxypoly[methyleneoxy]methyl-1-laza-3,7-dioxabicyclo[3.3.0]octane, quaternary adamantine or any combination thereof. In most examples, the preservative agent will be present in an amount of about 5-30 wt %. Moreover, it is generally contemplated that the preservative agents are free of chaotropic agents and/or detergents to reduce or avoid lysis of cells in contact with the preservative agents.

Suitable metabolic inhibitors may include glyceraldehyde, dihydroxyacetone phosphate, glyceraldehyde 3-phosphate, 1,3-bisphosphoglycerate, 3-phosphoglycerate, phosphoenolpyruvate, pyruvate, and glycerate dihydroxyacetate, and sodium fluoride, which concentration is typically in the range of between 0.1-10 wt %. Preferred chelators may include chelators of divalent cations, for example, ethylenediaminetetraacetic acid (EDTA) and/or ethylene glycol-bis(β-aminoethyl ether)-N,N,N′,N′-tetraacetic acid (EGTA), which concentration is typically in the range of between 1-15 wt %.

Additionally, RNA stabilizing reagent may further include protease inhibitors, phosphatase inhibitors and/or polyamines. Therefore, exemplary compositions for collecting and stabilizing ctRNA in whole blood may include aurintricarboxylic acid, diazolidinyl urea, glyceraldehyde/sodium fluoride, and/or EDTA. Further compositions and methods for ctRNA isolation are described in U.S. Pat. Nos. 8,304,187 and 8,586,306, which are incorporated by reference herein.

Most preferably, such contemplated RNA stabilization agents for ctRNA stabilization are disposed within a test tube that is suitable for blood collection, storage, transport, and/or centrifugation. Therefore, in most typical aspects, the collection tube is configured as an evacuated blood collection tube that also includes one or more serum separator substance to assist in separation of whole blood into a cell containing and a substantially cell free phase (no more than 1% of all cells present). In general, it is preferred that the RNA stabilization agents do not or substantially do not (e.g., equal or less than 1%, or equal or less than 0.1%, or equal or less than 0.01%, or equal or less than 0.001%, etc.) lyse blood cells. Viewed from a different perspective, RNA stabilization reagents will not lead to a substantial increase (e.g., increase in total RNA no more than 10%, or no more than 5%, or no more than 2%, or no more than 1%) in RNA quantities in serum or plasma after the reagents are combined with blood. Likewise, these reagents will also preserve physical integrity of the cells in the blood to reduce or even eliminate release of cellular RNA found in blood cell. Such preservation may be in form of collected blood that may or may not have been separated. In some aspects, contemplated reagents will stabilize ctRNA in a collected tissue other than blood for at 2 days, more preferably at least 5 days, and most preferably at least 7 days. Of course, it should be recognized that numerous other collection modalities other than collection tube (e.g., a test plate, a chip, a collection paper, a cartridge, etc.) are also deemed appropriate, and that the ctDNA and/or ctRNA can be at least partially purified or adsorbed to a solid phase to so increase stability prior to further processing.

As will be readily appreciated, fractionation of plasma and extraction of cfDNA and/or cfRNA can be done in numerous manners. In one exemplary preferred aspect, whole blood in 10 mL tubes is centrifuged to fractionate plasma at 1600 rcf for 20 minutes. The so obtained clarified plasma fraction is then separated and centrifuged at 16,000 rcf for 10 minutes to remove cell debris. Of course, various alternative centrifugal protocols are also deemed suitable so long as the centrifugation will not lead to substantial cell lysis (e.g., lysis of no more than 1%, or no more than 0.1%, or no more than 0.01%, or no more than 0.001% of all cells). ctDNA and ctRNA are extracted from 2mL of plasma using commercially available Qiagen reagents. For example, where cfRNA was isolated, the inventors used a second container that included a DNase that was retained in a filter material. Notably, the cfRNA also included miRNA (and other regulatory RNA such as shRNA, siRNA, and intronic RNA). Therefore, it should be appreciated that contemplated compositions and methods are also suitable for analysis of miRNA and other RNAs from whole blood.

Moreover, it should also be recognized that the extraction protocol was designed to remove potential contaminating blood cells, other impurities, and maintain stability of the nucleic acids during the extraction. All nucleic acids were kept in bar-coded matrix storage tubes, with ctDNA stored at −4° C. and ctRNA stored at −80° C. or reverse-transcribed to cDNA (e.g., using commercially reverse transcriptase such as Maxima or Superscript VILO) that is then stored at −4° C. or refrigerated at +2-8° C. Notably, so isolated ctRNA can be frozen prior to further processing.

It is contemplated that cfDNA and cfRNA may include any types of DNA/RNA that are originated or derived from tumor cells that are circulating in the bodily fluid of a person without being enclosed in a cell body or a nucleus. While not wishing to be bound by a particular theory, it is contemplated that release of cfDNA/cfRNA can be increased when the tumor cell interacts with an immune cell or when the tumor cells undergo cell death (e.g., necrosis, apoptosis, autophagy, etc.). Thus, in some embodiments, cfDNA/cfRNA may be enclosed in a vesicular structure (e.g., via exosomal release of cytoplasmic substances) so that it can be protected from nuclease (e.g., RNase) activity in some type of bodily fluid. Yet, it is also contemplated that in other aspects, the cfDNA/cfRNA is a naked DNA/RNA without being enclosed in any membranous structure, but may be in a stable form by itself or be stabilized via interaction with one or more non-nucleotide molecules (e.g., any RNA binding proteins, etc.).

Thus, the cfDNA may include any whole or fragmented genomic DNA, or mitochondrial DNA, and the cfRNA may include mRNA, tRNA, microRNA, small interfering RNA, long non-coding RNA (lncRNA). Most typically, the cell free DNA is a fragmented DNA typically with a length of at least 50 base pair (bp), 100 bp, 200 bp, 500 bp, or 1 kbp. Also, it is contemplated that the cfRNA is a full length or a fragment of mRNA (e.g., at least 70% of full-length, at least 50% of full length, at least 30% of full length, etc.).

Preferably, ctDNA/ctRNA may be derived from a gene including the patient- and tumor-specific mutation. Thus, in some embodiments, ctDNA/ctRNA may be a gene fragment that includes the at least a portion of the patient- and tumor-specific mutation. However, it is also contemplated that while the ctDNA/ctRNA is derived from the gene including the patient- and tumor-specific mutation, the ctDNA/ctRNA fragment may not include a whole or a portion of the patient- and tumor-specific mutation. In some embodiments, the ctDNA and ctRNA are fragments that may correspond to the same or substantially similar portion of the gene (e.g., at least 50%, at least 70%, at least 90% of the ctRNA sequence is complementary to ctDNA sequence, etc.). In other embodiments, the ctDNA and ctRNA are fragments may correspond to different portion of the gene (e.g., less than 50%, less than 30%, less than 20% of the ctRNA sequence is complementary to ctDNA sequence, etc.).

While less preferred, it is also contemplated that the ctDNA and cell free RNA may be derived from different genes from the tumor cell. In some embodiments, it is also contemplated that the ctDNA and cfRNA may be derived from different genes from the different types of cells (e.g., ctDNA from the tumor cell and cfRNA from the NK cell, etc.). In such scenarios, it is preferred that the ctDNA may include a whole or a portion of the patient- and tumor-specific mutation.

While ctDNA/ctRNA or cfRNA may include any type of DNA/RNA encoding any cellular, extracellular proteins or non-protein elements, it is preferred that at least some of ctDNA/ctRNA (or cfRNA from non-tumor cell) encodes one or more cancer-related proteins, inflammation-related proteins, DNA repair-related proteins, or RNA repair-related proteins, which mutation, expression and/or function may directly or indirectly be associated with tumorigenesis, metastasis, formation of immune suppressive tumor microenvironment, immune evasion, or presentation of patient-, tumor-specific neoepitope on the tumor cell. It is also contemplated that the ctDNA/ctRNA (or cfRNA from non-tumor cell) may be derived from one or more genes encoding cell machinery or structural proteins including, but not limited to, housekeeping genes, transcription factors, repressors, RNA splicing machinery or elements, translation factors, tRNA synthetases, RNA binding protein, ribosomal proteins, mitochondrial ribosomal proteins, RNA polymerase, proteins related to protein processing, heat shock proteins, cell cycle-related proteins, elements related to carbohydrate metabolism, lipid, citric acid cycle, amino acid metabolism, NADH dehydrogenase, cytochrome c oxidase, ATPase, lysosome, proteasome, cytoskeletal proteins and organelle synthesis.

In an especially preferred embodiment, contemplated ctRNAs include those that encode tumor associated antigens, tumor specific antigens, overexpressed RNA (where the RNA is expressed at a higher level than in a non-tumor cell), RNA that includes a patient and tumor specific mutation, and particularly where the mutation encodes a neoepitope (i.e., mutation is part of a codon that results in a changed amino acid). In especially contemplated aspects, it should be appreciated that patient and tumor specific mutations, and especially neoepitope mutations are advantageous in treatment and monitoring of treatment where the patient is treated with a neoepitope based therapeutic composition (e.g., DNA plasmid vaccine, yeast, or viral expression system). Moreover, suitable ctRNA also include all sequences that are known or suspected protooncogenes and/or oncogenes (tumor promoter or tumor suppressor). Thus, contemplated oncogenes include those that encode one or more growth factors, encode a protein that forms part of a signal transduction network (e.g., tyrosine kinases, serine or threonine kinases, GTPases, etc.), and/or encode a protein that operates as transcription factor or is involved in cell cycle regulation or DNA repair.

For example, where a cancer is associated with one or more mutation in ras, it is contemplated that suitable ctRNA assays may detect and/or quantify mutated ras sequences, and especially contemplated ras mutations include mutations at amino acid positions 12, 13, and 61 (e.g., G12A, G12C, G12D, G12R, G12S, G12V, G13A, G13C, G13D, G13R, G13S, G13V, Q61E, Q61H, Q61K, Q61L, Q61P, and Q61R) in h-ras, n-ras, and k-ras. Further contemplated mutations in ras include all known oncogenic mutations, and exemplary mutations are disclosed in WO2015/123532 and Nature Reviews Drug Discovery 13, 828-851 (2014), incorporated by reference herein. Other ctRNA include sequences encoding EGFR, ALK fusion, and ROS1. Selection of suitable ctRNA may be based on molecular profiling of a patient's omics data, and/or on presence of known mutant sequences commonly found in specific cancers.

In another preferred embodiment, suitable ctRNAs may also include those that are involved with immune stimulation and/or immune suppression. For example, NKD2D ligands (and especially soluble NKG2D ligands such as MICA) are known to reduce cytotoxic activity of NK cells and CTLs, and detection and/or quantification of ctRNA encoding NKG2D ligands (and especially soluble NKG2D ligands) is therefore especially contemplated. Similarly, and as discussed in more detail below, other ctRNA that encode various immune modulatory factors, including PD-1L are also deemed suitable. Suitable ctRNA molecules may also encode proteins that indirectly down-regulate an anti-tumor immune response, and contemplated ctRNAs thus include those encoding MUC1. In further examples, ctRNA that encode various cancer hallmark genes are contemplated. For example, where the hallmark is EMT (epithelial-mesenchymal transition), contemplated ctRNA may encode brachyury. In these and other cases (especially where secreted inhibitory factors are present), it is contemplated that upon detection of the ctRNA suitable therapeutic action may be taken (e.g., apheretic removal of such soluble factors, etc.).

It is also contemplated that ctDNA/ctRNA or cfRNA may present in modified forms or different isoforms. For example, the ctDNA may be present in methylated or hydroxyl methylated, and the methylation level of some genes (e.g., GSTP1, p16, APC, etc.) may be a hallmark of specific types of cancer (e.g., colorectal cancer, etc.). The ctRNA may be present in a plurality of isoforms (e.g., splicing variants, etc.) that may be associated with different cell types and/or location. Preferably, different isoforms of ctRNA may be a hallmark of specific tissues (e.g., brain, intestine, adipose tissue, muscle, etc.), or may be a hallmark of cancer (e.g., different isoform is present in the cancer cell compared to corresponding normal cell, or the ratio of different isoforms is different in the cancer cell compared to corresponding normal cell, etc.). For example, mRNA encoding HMGB1 are present in 18 different alternative splicing variants and 2 unspliced forms. Those isoforms are expected to express in different tissues/locations of the patient's body (e.g., isoform A is specific to prostate, isoform B is specific to brain, isoform C is specific to spleen, etc.). Thus, in these embodiments, identifying the isoforms of ctRNA in the patient's bodily fluid can provide information on the origin (e.g., cell type, tissue type, etc.) of the ctRNA.

Alternatively or additionally, the inventors contemplate ctRNA may include regulatory noncoding RNA (e.g., microRNA, small interfering RNA, long non-coding RNA (lncRNA)), which quantities and/or isoforms (or subtypes) can vary and fluctuate by presence of a tumor or immune response against the tumor. Without wishing to be bound by any specific theory, varied expression of regulatory noncoding RNA in a cancer patient's bodily fluid may due to genetic modification of the cancer cell (e.g., deletion, translocation of parts of a chromosome, etc.), and/or inflammations at the cancer tissue by immune system (e.g., regulation of miR-29 family by activation of interferon signaling and/or virus infection, etc.). Thus, in some embodiments, the ctRNA can be a regulatory noncoding RNA that modulates expression (e.g., downregulates, silences, etc.) of mRNA encoding a cancer-related protein or an inflammation-related protein (e.g., HMGB1, HMGB2, HMGB3, MUC1, VWF, MMP, CRP, PBEF1, TNF-α, TGF-β, PDGFA, IL-1, IL-2, IL-3, IL-4, IL-5, IL-6, IL-7, IL-8, IL-9, IL-10, IL-12, IL-13, IL-15, IL-17, Eotaxin, FGF, G-CSF, GM-CSF, IFN-γ, IP-10, MCP-1, PDGF, hTERT, etc.).

It is also contemplated that some cell free regulatory noncoding RNA may be present in a plurality of isoforms or members (e.g., members of miR-29 family, etc.) that may be associated with different cell types and/or location. Preferably, different isoforms or members of regulatory noncoding RNA may be a hallmark of specific tissues (e.g., brain, intestine, adipose tissue, muscle, etc.), or may be a hallmark of cancer (e.g., different isoform is present in the cancer cell compared to corresponding normal cell, or the ratio of different isoforms is different in the cancer cell compared to corresponding normal cell, etc.). For example, higher expression level of miR-155 in the bodily fluid can be associated with the presence of breast tumor, and the reduced expression level of miR-155 can be associated with reduced size of breast tumor. Thus, in these embodiments, identifying the isoforms of cell free regulatory noncoding RNA in the patient's bodily fluid can provide information on the origin (e.g., cell type, tissue type, etc.) of the cell free regulatory noncoding RNA.

Once ctDNA/ctRNA or cfRNA is isolated, various types of omics data can be obtained using any suitable methods. DNA sequence data will not only include the presence or absence of a gene that is associated with cancer or inflammation, but also take into account mutation data where the gene is mutated, the copy number (e.g., to identify duplication, loss of allele or heterozygosity), and epigenetic status (e.g., methylation, histone phosphorylation, nucleosome positioning, etc.). With respect to RNA sequence data it should be noted that contemplated RNA sequence data include mRNA sequence data, splice variant data, polyadenylation information, etc. Moreover, it is generally preferred that the RNA sequence data also include a metric for the transcription strength (e.g., number of transcripts of a damage repair gene per million total transcripts, number of transcripts of a damage repair gene per total number of transcripts for all damage repair genes, number of transcripts of a damage repair gene per number of transcripts for actin or other household gene RNA, etc.), and for the transcript stability (e.g., a length of poly A tail, etc.).

With respect to the transcription strength (expression level), transcription strength of the ctRNA or cfRNA can be examined by quantifying the ctRNA or cfRNA. Quantification of V can be performed in numerous manners, however, expression of analytes is preferably measured by quantitative real-time RT-PCR of ctRNA or cfRNA using primers specific for each gene. For example, amplification can be performed using an assay in a 10 μL reaction mix containing 2 μL ctRNA or cfRNA, primers, and probe. mRNA of α-actin or β-actin can be used as an internal control for the input level of ctRNA or cfRNA. A standard curve or single reaction of samples with known concentrations of each analyte was included in each PCR plate as well as positive and negative controls for each gene. Test samples were identified by scanning the 2D barcode on the matrix tubes containing the nucleic acids. Delta Ct (dCT) was calculated from the Ct value derived from quantitative PCR (qPCR) amplification for each analyte subtracted by the Ct value of actin for each individual patient's blood sample. Relative expression of patient specimens is calculated using a standard curve of delta Cts of serial dilutions of Universal Human Reference RNA or another control known to express the gene of interest set at a gene expression value of 10 or a suitable whole number allowing for a range of patient sample results for the specific to be resulted in the range of approximately 1 to 1000 (when the delta CTs were plotted against the log concentration of each analyte). Alternatively and/or additionally, Delta Cts vs. log₁₀Relative Gene Expression (standard curves) for each gene test can be captured over hundreds of PCR plates of reactions (historical reactions). A linear regression analysis can be performed for each assays and used to calculate gene expression from a single point from the original standard curve going forward.

Alternatively or additionally, where discovery or scanning for new mutations or changes in expression of a particular gene is desired, real time quantitative PCR may be replaced by or added with RNAseq to so cover at least part of a patient transcriptome. Moreover, it should be appreciated that analysis can be performed static or over a time course with repeated sampling to obtain a dynamic picture without the need for biopsy of the tumor or a metastasis.

In addition to RNA quantification, RNA sequencing of the cfRNA (directly or via reverse transcription) may be performed to verify identity and/or identify post-transcriptional modifications, splice variations, and/or RNA editing. To that end, sequence information may be compared to prior RNA sequences of the same patient (of another patient, or a reference RNA), preferably using synchronous location guided analysis (e.g., using BAMBAM as described in US Pat. Pub. No. 2012/0059670 and/or US2012/0066001). Such analysis is particularly advantageous as such identified mutations can be filtered for neoepitopes that are unique to the patient, presented in the MHC I and/or II complex of the patient, and as such serve as therapeutic target. Moreover, suitable mutations may also be further characterized using a pathway model and the patient- and tumor-specific mutation to infer a physiological parameter of the tumor. For example, especially suitable pathway models include PARADIGM (see e.g., WO 2011/139345, WO 2013/062505) and similar models (see e.g., WO 2017/033154). Moreover, suitable mutations may also be unique to a sub-population of cancer cells. Thus, mutations may be selected based on the patient and specific tumor (and even metastasis), on the suitability as therapeutic target, type of gene (e.g., cancer driver gene, etc.), and affected function of the gene product encoded by the gene with the mutation.

Moreover, the inventors contemplate that multiple types of cfDNA and/or cfRNA can be isolated, detected and/or quantified from the same bodily fluid sample of the patient such that the relationship or association among the mutation, quantity, and/or subtypes of multiple cfDNA and/or cfRNA can be determined for further analysis. Thus, in one embodiment, from a single bodily fluid sample or from a plurality of bodily fluid samples obtained in a substantially similar time points, from a patient, multiple cfRNA species can be detected and quantified. In this embodiment, it is especially preferred that at least some of the cfRNA measurements are specific with respect to a cancer associated nucleic acid.

Consequently, such obtained omics data information of ctDNA/ctRNA or cfRNA along with information of tumor-specific, patient-specific mutation in one or more gene can be used for diagnosis of tumor, monitoring of prognosis of the tumor, monitoring the effectiveness of treatment provided to the patients, evaluating a treatment regime based on a likelihood of success of the treatment regime, and even as discovery tool that allows repeated and non-invasive sampling of a patient.

For example, early detection of cancer, regardless specific anatomical or molecular type of tumor, can be achieved by measuring overall quantity of ctDNAs and/or ctRNAs in the sample of the patient's bodily fluid (as e.g., described in International Patent Application PCT/US18/22747, incorporated by reference herein). It is contemplated that presence of cancer in the patient can be assumed or inferred when overall cfDNA and/or cfRNA quantity reaches a particular or predetermined threshold. The predetermined threshold of cfDNA and/or cfRNA quantity can be determined by measuring overall cfDNA and/or cfRNA quantity from a plurality of healthy individuals in a similar physical condition (e.g., ethnicity, gender, age, other predisposed genetic or disease condition, etc.).

For example, predetermined threshold of cfDNA and/or cfRNA quantity is at least 20%, at least 30%, at least 40%, at least 50% more than the average or median number of cfDNA and/or cfRNA quantity of healthy individual. It should be appreciated that such approach to detect tumor early can be performed without a priori knowledge on anatomical or molecular characteristics or tumor, or even the presence of the tumor. To further obtain cancer specific information and/or information about the status of the immune system, additional cfRNA markers may be detected and/or quantified. Most typically, such additional cfRNA markers will include cfRNA encoding one or more oncogenes as described above and/or one or more cfRNA encoding a protein that is associated with immune suppression or other immune evading mechanism. Among other markers in such use, particularly contemplated cfRNAs include those encoding MUC1, MICA, brachyury, and/or PD-L1.

The inventors further contemplate that once the tumor is identified or detected, the prognosis of the tumor can be monitored by monitoring the types and/or quantity of cfDNAs and/or cfRNAs in various time points. As described, a patient- and tumor-specific mutation is identified in a gene of a tumor of the patient. Once identified, cfDNAs and/or cfRNAs, at least one of which comprises the patient- and tumor-specific mutation, are isolated from a bodily fluid of the patient (typically whole blood, plasma, serum), and then the mutation, quantity, and/or subtype of cfDNAs and/or cfRNAs are detected and/or quantified. The inventors contemplate that the mutation, quantity, and/or subtype of cfDNAs and/or cfRNAs detected from the patient's bodily fluid can be a strong indicator of the state, size, and location of the tumor. For example, increased quantity of cfDNAs and/or cfRNAs having a patient- and tumor-specific mutation can be an indicator of increased tumor cell lysis upon immune response against the tumor cell and/or increased numbers of tumor cells having the mutation. In another example, increased ratio of cfRNA over cfDNA having the patient- and tumor-specific mutation (where cfRNA and cfDNA are derived from the same gene having the mutation) may indicate that such patient- and tumor-specific mutation may cause increased transcription of the mutated gene to potentially trigger tumorigenesis or affects the tumor cell function (e.g., immune-resistance, related to metastasis, etc.). In still another example, increased quantity of a ctRNA having a patient- and tumor-specific mutation along with increased quantity of another ctRNA (or non-tumor related cfRNA) may indicate that the another ctRNA may be in the same pathway with the ctRNA having a patient- and tumor-specific mutation such that the expression or activity of two ctRNA (or a ctRNA and a cfRNA) may be correlated (e.g., co-regulated, one affect another, one is upstream of another in the pathway, etc.).

Thus, it should be appreciated that the results from cfRNA quantification can not only be used as an indicator for the presence or absence of a specific cell or population of cells that gave rise to the measured cfRNA, but can also serve as an additional indicator of the state (e.g., genetic, metabolic, related to cell division, necrosis, and/or apoptosis) of such cells or population of cells, particularly where the results from cfRNA quantification are employed as input data in pathway analysis and/or machine learning models. For example, suitable models include those that predict pathway activity (or activity of components of a pathway) in a single or multiple pathways. Thus, quantified cfRNA may also be employed as input data into models and modeling systems in addition to or as replacement for RNA data from transcriptomic analysis (e.g., obtained via RNAseq or cDNA or RNA arrays).

In particularly preferred aspects, ctDNA/ctRNA or cfRNA may include nucleic acid sequence encoding a neoepitope that is also a suitable target for immune therapy. Without wishing to be bound by any specific theory, the inventors contemplate that a gene with a patient- and tumor-specific mutation is likely to generate a neoepitope if the quantity of ctRNA derived from the gene is increased (e.g., at least 20%, at least 40%, at least 50%, etc.) in the patients upon developing the tumor. Based on the gene sequences having the patient- and tumor-specific mutation, a sequence of potential neoepitope can be generated, which can then be further filtered for a match to the patient's HLA type to thereby increase likelihood of antigen presentation of the neoepitope. Most preferably, such matching can be done in silico. Most typically, the patient-specific epitopes are unique to the patient, but may also in at least some cases include tumor type-specific neoepitopes (e.g., Her-2, PSA, brachyury, etc.) or cancer-associated neoepitopes (e.g., CEA, MUC-1, CYPB1, etc.). Any suitable immune therapies targeting the neoepitope are contemplated, and the exemplary immune therapies may include an antibody-based immune therapy targeting the neoepitope with a binding molecule (e.g., antibody, a fragment of antibody, an scFv, etc.) to the neoepitope and a cell-based immune therapy (e.g., an immune competent cell having a receptor specific to the neoepitope, etc.). For example, the cell-based immune therapy may include a T cell, NK cell, and/or NKT cells expressing a chimeric antigen receptor specific to the neoepitope derived from the gene having the patient- and tumor-specific mutation.

Additionally, it is also contemplated that the ctDNAs and/or ctRNAs can be detected, quantified and/or analyzed over time (at different time points) to determine the progress/prognosis of the tumor and/or determine the effectiveness of a treatment to the patient. Generally, multiple measurements can be obtained over time from the same patient and same bodily fluid, and at least a first ctRNA may be quantified at a single time point or over time. Most preferably, such first ctRNA is from a tumor associated gene, a tumor specific gene, or covers a patient- and tumor specific mutation. Over at least one other time point, a second cfRNA may then be quantified, and the first and second quantities may then be correlated for diagnosis and/or monitoring treatment. Alternatively, the second cfRNA may also be derived from a gene that is relevant to the immune status of the patient. For example, suitable cfRNAs may be derived from a checkpoint inhibition related gene, a cytokine related gene, and/or a chemokine related gene, or the second cfRNA is a miRNA. Thus, contemplated systems and methods will not only allow for monitoring of a specific gene, but also for the status of an immune system. For example, where the second cfRNA is derived from a checkpoint receptor ligand or IL-8 gene, the immune system may be suppressed. On the other hand, where the second cfRNA is derived from an IL-12 or IL-15 gene, the immune system may be activated. Thus, measurement of a second cfRNA may further inform treatment. Likewise, the second cfRNA may also be derived from a second metastasis or a subclone, and may be used as a proxy marker for treatment efficacy. In this regard, it should also be noted that the efficacy of immune therapy can be indirectly monitored using contemplated systems and methods. For example, where the patient was vaccinated with a DNA plasmid, recombinant yeast, or adenovirus, from which a neoepitope or polytope is expressed, cfRNA of such recombinant vectors may be detected and as such validate transcription from these recombinant vectors.

Particularly where the cfRNA is quantified over time, it is generally preferred that more than one measurement of the same (and in some cases newly identified) mutation are performed. For example, multiple measurements over time may be useful in monitoring treatment effect that targets the specific mutation or neoepitope. Thus, such measurements can be performed before/during and/or after treatment. Where new mutations are detected, such new mutations will typically be located in a different gene and as such multiple and distinct cfRNAs are monitored.

Regardless of the type and number of mutations, it is generally preferred that a patient record is generated or updated with an indication that is associated with a quantity of the cfRNA and/or that a treatment option is associated with a particular measured amount of quantified cfRNA and/or that effectiveness of a treatment (e.g., immune therapy, radiotherapy, chemotherapy, etc.) to the tumor. Moreover, the patient records can also be established for a specific disease (e.g., particular cancer, or sub-type of cancer), a specific disease parameter (e.g., treatment resistant to specific drug, sensitive to a drug), or disease associated state (e.g., responsive to immune stimulants such as cytokines or checkpoint inhibitors). Viewed from a different perspective, it should therefore also be recognized that the cfRNA results may be patient-specific, or specific to a particular disease, disease parameter, or disease associated state, and as such also qualify as a cohort-specific parameter.

Thus, it should be appreciated that cfRNA of a patient can be identified, quantified, or otherwise characterized in any appropriate manner. For example, it is contemplated that systems and methods related to blood-based RNA expression testing (cfRNA) that identify, quantify expression, and allow for non-invasive monitoring of changes in drivers of disease (e.g., PD-L1 and nivolumab or pembrolizumab) be used, alone or in combination with analysis of biopsied tissues. Such cfRNA centric systems and methods allow monitoring changes in drivers of a disease and/or to identify changes in drug targets that may be associated with emerging resistance to chemotherapies. For example, cfRNA presence and/or quantity of one or more specific gene (e.g., mutated or wild-type, from tumor tissue and/or T-lymphocytes) may be used as a diagnostic tool to assess whether or not a patient may be sensitive to one or more checkpoint inhibitors, such as may be provided by analysis of cfRNA for ICOS signaling.

Moreover, and viewed from yet another perspective, the inventors also contemplate that contemplated systems and methods may be employed to generate a mutational signature of a tumor in a patient. In such method, one or more cfRNAs are quantified where at least one of the genes leading to those cfRNAs comprises a patient- and tumor-specific mutation. Such signature may be particularly useful in comparison with a mutational signature of a solid tumor, especially where both signatures are normalized against healthy tissue of the same patient. Differences in signatures may be indicative of treatment options and/or likelihood of success of the treatment options. Moreover, such signatures may also be monitored over time to identify subpopulations of cells that appear to be resistant or less responsive to treatment. Such mutational signatures may also be useful in identifying tumor specific expression of one or more proteins, and especially membrane bound or secreted proteins, that may serve as a signaling and/or feedback signal in AND/NAND gated therapeutic compositions. Such compositions are described in copending U.S. application with the Ser. No. 15/897,816, which is incorporated by reference herein.

Among various other advantages, it should be appreciated that use of contemplated systems and methods simplifies treatment monitoring and even long term follow-up of a patient as target sequences are already pre-identified and target cfRNA can be readily surveyed using simple blood tests without the need for a biopsy. Such is particularly advantageous where micro-metastases are present or where the tumor or metastasis is at a location that precludes biopsy. Further, it should be also appreciated that contemplated compositions and methods are independent of a priori knowledge on known mutations leading to or associated with a cancer. Still further, contemplated methods also allow for monitoring clonal tumor cell populations as well as for prediction of treatment success with an immunomodulatory therapy (e.g., checkpoint inhibitors or cytokines), and especially with neoepitope-based treatments (e.g., using DNA plasmid vaccines and/or viral or yeast expression systems that express neoepitopes or polytopes).

With respect to preventative and/or prophylactic use, it is contemplated that identification and/or quantification of known cfDNAs and/or cfRNAs may be employed to assess the presence or risk of onset of cancer (or other disease or presence of a pathogen). Depending on the particular cfRNA detected, it is also contemplated that the cfDNAs and/or cfRNAs may provide guidance as to likely treatment outcome with a specific drug or regimen (e.g., surgery, chemotherapy, radiation therapy, immunotherapeutic therapy, dietary treatment, behavior modification, etc.). Similarly, quantitative cfRNA results may be used to gauge tumor health, to modify immunotherapeutic treatment of cancer in patient (e.g., to quantify sequences and change target of treatment accordingly), or to assess treatment efficacy. The patient may also be placed on a post-treatment diagnostic test schedule to monitor the patient for a relapse or change in disease and/or immune status.

Thus, the inventors further contemplate that, based on cfDNAs and/or cfRNAs detected, analyzed, and/or quantified, a new treatment plan can be generated and recommended or a previously used treatment plan can be updated. For example, a treatment recommendation to use immunotherapy to target a neoepitope encoded by gene A can be provided based on the detection of ctDNA and/or ctRNA (derived from gene A) and increased expression level of ctRNA having patient-and tumor-specific mutation in gene A, which is obtained from the patient's first blood sample. After 1 month of treatment with an antibody targeting the neoepitope encoded by gene A, the second blood sample was drawn, and ctRNA levels were determined. In the second blood sample, ctRNA expression level of gene A is decreased while ctRNA expression level of gene B is increased. Based on such updated result, a treatment recommendation can be updated to target neoepitope encoded by gene B. Also, the patient record can be updated that the treatment targeting the neoepitope encoded by gene A was effective to reduce the number of tumor cells expressing neoepitope encoded by gene A.

### EXAMPLES

Based on the unmet need to evaluate tumor response by means other than radiology tests, the inventors contemplated measurements of changes in gene expression, allele-fractions of mutations, PDL-1 expression and/or quantities of cell free DNA [ctDNA] and/or RNA [ctRNA] in the plasma of patients to monitor disease state and to predict outcome to anti-tumoral therapy.

Isolation of ctDNA/ctRNA from whole blood: Whole blood was obtained by venipuncture and 10 ml were collected into cell-free RNA BCT® tubes or cell-free DNA BCT® tubes (Streck Inc., 7002 S. 109^{th} St., La Vista Nebr. 68128) containing RNA or DNA stabilizers, respectively. The sample tubes were then centrifuged at 1,600 rcf for 20 minutes, plasma was withdrawn and further centrifuged at 16,000 rcf for 10 minutes to remove cell debris. Plasma was used to isolate cfRNA using commercially available RNA isolation kits following the manufacturer's protocol with slight modification. Specifically, DNA was removed from the sample in an on-column DNAse digest. In an alternative approach, cfRNA was also obtained in an automated manner using a robotic extraction method on QiaSymphony™ instrumentation (Qiagen, 19300 Germantown Road; Germantown, Md. 20874), slightly modified to accommodate for DNA removal where desired. The robotic extraction maintains approximately 12% DNA contamination (less than 25% is our cut-off for quality purposes) in the cfRNA sample. The inventors found that 25% DNA contamination does not affect our PCR results as the inherent error in PCR is two-fold. We measured the relative expression of Excision Repair Cross-Complementing enzyme (ERCC1) vs beta action the same twenty-one NSCLC samples to determine whether there was a significant difference between the two extraction procedures. There was no statistical difference in the relative expression generated by the new process and the previous process using PCR technology. p=0.4111 (paired t-test) Note: a statistically difference would have been p<0.05 for this test.

In one example, the inventors measured serial levels of plasma ctDNA/ctRNA in metastatic patients with non-small cell lung cancer (NSCLC) and breast cancers undergoing first line treatment and correlated them with response (complete response (CR)/partial response (PR)/stable disease (SD)/progressive disease (PD)) seen by CT scans. The inventors also monitored PD-L1 expression in NSCLC patients treated with immunotherapy. ctDNA and ctRNA were extracted from plasma, ctRNA was reverse transcribed with random primers to cDNA. Quantities of ctDNA and ctRNA were then determined by RT-qPCR.

More specifically, 52 patients were enrolled (28 breast/24 NSCLC) in this experiment in two separate patient groups: 28 patients in Breast group and 24 patients in NSCLC group. In Breast group, 39% (11/28) were Caucasian (NHW) and 36% (10/28) were Hispanic (H), and 20 patients out of 52 patients completed therapy. 2 patients had PR and showed no change (NC) or decrease (DEC) in levels of ctDNA/ctRNA. 11 patients achieved SD, 9 had NC levels of ctDNA/ctRNA. Among patients with PD, 5 out of 5 patients underwent significant increase (INC) in ctDNA/ctRNA levels. Overall, among breast patients, there was an 84% (16/19) agreement between response and levels of ctDNA/ctRNA. These were correlated with one another (r=0.7002, p<0.0001). In NSCLC group, 71% (16/24) were NHW and 25% (6/24) H. Among all, 87% (21/24) of patients had non-squamous cell carcinoma (SQCC). Out of 20 patients who had CT scans, one pt had PR with DEC levels of ctDNA/ctRNA, 10 patients achieved SD, who all showed DEC or NC levels of ctDNA/ctRNA. 8 patients had PD, 6 of them had INC in ctDNA/ctRNA levels even 7 weeks prior to PD. Among NSCLC patients, there was a 90% (17/19) agreement in response and levels of ctDNA/ctRNA. These were correlated with one another (r=0.6231, p<0.0001). In 5 patients, PD-L1 expression remained stable when CT scans showed SD or PR.

As can be seen Table 4, there is a strong correlation between clinical responses with changes in plasma levels of ctDNA/ctRNA in patients with NSCLC (90%) and breast cancer (84%). Some of these could be documented weeks before imaging was done. Thus, ctRNA can be used as effective as ctDNA as a predictive tool.

To further confirm validity of ctRNA and ctDNA results, the inventors performed a concordance assay in which tissue biopsy values and liquid biopsy results were compared in a double blinded test for two cancer types. Notably, and as shown in the Table below, the data correlated very well and established the utility of ctRNA and ctDNA as prognostic and diagnostic markers.

TABLE 4

| CRC (Tissue) | | | NSCLC (Tissue) | | |
|---|---|---|---|---|---|
| BRAF: V600E (5/48) | | | EGFR: ex19dels (6/183), L858R | | |
| KRAS: G12A (1/61), G12D (9/61), | | | (3/185), T790M (28/185) | | |
| G12S (3/61), G12V (7/61), G13D (2/61) | | | KRAS: G12C (20/181), G12D (5/178), | | |
| NRAS: Q61R (1/31) | | | G12V (9/182) | | |
| CRC (Liquid Biopsy) | | | PD-L1: S/12 | | |
| BRAF: V600E (5/48) | | | NSCLC (Liquid Biopsy) | | |
| KRAS: G12A (1/61), G12D (9/61), | | | EGFR: ex19dels (5/183), L858R | | |
| G12S (2/61), G12V (4/61), G13D (2/61) | | | (3/185), T790M (38/185) | | |
| NRAS: Q61R (1/31) | | | KRAS: G12C (20/181), G12D (7/178), | | |
| | | | G12V (9/182) | | |
| | | | PD-L1: 7/12 | | |
| | | % Agreement | | | % Agreement |
| | | (tissue vs. | | | (tissue vs. |
| Gene | Variant | liquid biopsy | Gene | Variant | liquid biopsy |
| BRAF | V600E | 100% | EGFR | L858R | 100% |
| KRAS | G12A | 100% | | Ex19del | 95.5% |
| | G12D | 93% | | T790M | 94.7% |
| | G12S | 98% | KRAS | G12C | 98.9% |
| | G12V | 95.1% | | G12D | 98.9% |
| | G13D | 100% | | G12V | 100% |
| NRAS | Q61R | 100% | PD-L1 | WT | 83.3% |

In yet another example, FOLFOXIRI plus Bevacizumab has been used as a standard initial therapy for metastatic colorectal cancer (mCRC) and should be one of preferred regimens in tumors with RAS mutation. However, frequent febrile neutropenia (FN) was reported in Japanese patients receiving the FOLFOXIRI plus Bevacizumab. The inventors performed a phase II trial to assess the safety and activity of 1st-line m-FOLFOXIRI plus Bevacizumab for RAS mutation in mCRC, which was accompanied by the liquid biopsy (LB) research (UMIN000015152).

Specifically, patients with unresectable/measurable tumors with RAS mutation tumors, were given the combination of Bevacizumab and m-FOLFOXIRI (irinotecan 150 mg/m², oxaliplatin 85 mg/m², and levofolinate [LV] 200 mg/m², and fluorouracil 2400 mg/m² and repeated biweekly). After induction therapy of a maximum of 12 cycles, maintenance therapy with fluorouracil/leucovorin plus Bevacizumab was administered. The primary endpoint was objective response rate (ORR). Progression-free survival (PFS), overall survival, early tumor shrinkage (ETS), depth of response (DpR), and safety were secondary endpoints. Plasma samples were collected at 3 points (pre-, 8w, and progression) during treatment. Target ctDNA mutations were tested for on qPCR using Competitive Allele-Specific TaqMan® PCR assays specific for KRAS, NRAS, BRAF, and PIK3CA variants.

Sixty-two of 64 participants were evaluable for efficacy of the FOLFOXIRI plus Bevacizumab. The media age of participants group was 63 year old (36-75). The 55% of the participants were male, and 45% were female. 92% of the participants were in favorable PSO stage, and 27% of the participants had a right-sided tumor. Median follow-up time was 7.9 months. Objective response rate (ORR) and disease control rate were 74.2% and 96.8%, respectively. Among the participants, 74% of participants showed ETS and median DpR was 48%. Median PFS was not reached. Common grade 3 or 4 adverse events were neutropenia (49%), hypertension (22%), diarrhea (13%), and FN (4.8%). No treatment-related deaths occurred. Liquid biopsy study showed that any mutation was observed in 72% (38/53) patients at pre-treatment. The presence of mutation at 8 weeks was correlated with ORR regardless of mutation status at pre-treatment [no mutation; 80% (32/40), any mutation; 45% (5/11), P=0.05]. Moreover, patients with PIK3CA mutation at pre-treatment had a poor response (43%, 3/7).

It was observed that m-FOLFOXIRI plus Bevacizumab is active without impacting efficacy for RAS mutated mCRC and may be more feasible for Japanese patients. The status of KRAS, NRAS, PIK3CA mutation may potentially predict best response to triplet plus Bevacizumab.

In addition to cancer, contemplated systems and methods are also useful for various other test systems that rely on the presence and/or quantity of specific markers. Consequently, the methods presented herein may be adopted to background/substance abuse tests, screening for immigration, travel, or pandemic control, and screening for identification of insurance risk. Further considerations and embodiments are provided in copending PCT application with the serial number PCT/US18/22747, and WO 2016/077709, which are incorporated by reference herein.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus

1. A method of monitoring a cancer in a patient, comprising:
identifying a patient- and tumor-specific mutation in a gene of a tumor of the patient;
obtaining a bodily fluid of the patient; and
quantifying cfRNA comprising the patient- and tumor-specific mutation in the bodily fluid of the patient.

2. The method of claim 1, wherein the step of identifying comprises comparing omics data from tumor tissue and normal tissue of the same patient.

3. The method of claim 2, wherein the omics data include whole genome sequence data, exome sequence data, transcriptome sequence data, and/or proteome sequence data.

4. The method of claim 2, wherein the omics data are compared in an incremental synchronous manner.

5. The method of claim 1, further comprising a step of using a pathway model and the patient- and tumor-specific mutation to infer a physiological parameter of the tumor.

6. The method of claim 5, wherein the pathway model is PARADIGM, and optionally wherein the physiological parameter is sensitivity of the tumor to a drug.

7. The method of claim 1, wherein the patient- and tumor-specific mutation encodes a neoepitope.

8. The method of claim 1, wherein the patient- and tumor-specific mutation is located in a cancer driver gene.

9. The method of claim 1, further comprising a step of associating the patient- and tumor-specific mutation with a clonal population of cancer cells within the tumor.

10. The method of claim 1, wherein the step of obtaining the bodily fluid and the step of quantifying the cfRNA are repeated.

11. The method of claim 10, wherein the steps are repeated during treatment of the patient.

12. The method of claim 10, wherein the steps are repeated after treatment of the patient.

13. The method of claim 1, wherein the step of identifying the patient- and tumor-specific mutation is repeated during or after treatment of the patient and identifies a second patient- and tumor-specific mutation in a second gene.

14. The method of claim 13, further comprising a step of quantifying a cfRNA comprising the second patient- and tumor-specific mutation.

15. The method of claim 1, wherein the cfRNA comprises a miRNA.

16. (canceled)

17. The method of claim 1, wherein the step of quantifying includes real time quantitative PCR of a cDNA prepared from the cfRNA.

18. The method of claim 1, further comprising a step of archiving at least some of the bodily fluid or cfRNA isolated from the bodily fluid or cDNA prepared from the cfRNA, and optionally wherein the step of archiving the cfRNA comprises freezing at −80° C. or wherein the step of archiving the cDNA comprises freezing at −4° C. or storing at +2-8° C.

19-20. (canceled)

21. A method of monitoring a cancer in a patient, comprising:
obtaining a plurality of bodily fluids of the patient at a plurality of respective time points; and
quantifying a first cfRNA in each of the bodily fluids of the patient, wherein the first cfRNA comprises a first patient- and tumor-specific mutation in a gene of a tumor of the patient.

22-57. (canceled)

58. A method of isolating cfRNA, comprising:
centrifuging whole blood at a first RCF to obtain a plasma fraction, and centrifuging the plasma fraction at a second RCF to obtain a clarified plasma fraction; and
subjecting at least a portion of the clarified plasma fraction to a DNA degradation step to degrade cfDNA and gDNA.

59-78. (canceled)
